SEQUENCE LISTING (12 sequences)

SEQ ID NO: 1 - DNA, 36 bp, Artificial Sequence
  Synthetic oligonucleotide primer
acacggccgtgtattactgtgcgagagaaaaaaaaa

SEQ ID NO: 2 - DNA, 8 bp, Artificial Sequence
  Synthetic oligonucleotide primer
gggggggg

SEQ ID NO: 3 - DNA, 8 bp, Artificial Sequence
  Synthetic oligonucleotide primer
cccccccc

SEQ ID NO: 4 - DNA, 8 bp, Artificial Sequence
  Synthetic oligonucleotide primer
tttttttt

SEQ ID NO: 5 - DNA, 32 bp, Artificial Sequence
  Synthetic oligonucleotide primer
acacggccgtgtattactgtgcnnnnnnacgt

SEQ ID NO: 6 - DNA, 36 bp, Artificial Sequence
  Synthetic oligonucleotide primer
acacggccgtgtattactgtgcnnnnnnacgtaaaa

SEQ ID NO: 7 - DNA, 14 bp, Artificial Sequence
  Synthetic oligonucleotide primerr
nnnnnnacgtgggg

SEQ ID NO: 8 - DNA, 14 bp, Artificial Sequence
  Synthetic oligonucleotide primer
nnnnnnacgtcccc

SEQ ID NO: 9 - DNA, 14 bp, Artificial Sequence
  Synthetic oligonucleotide primer
nnnnnnacgttttt

SEQ ID NO: 10 - DNA, 36 bp, Artificial Sequence
  Synthetic oligonucleotide primer
acacggccgtgtattactgtgcggggggnnnntcag

SEQ ID NO: 11 - DNA, 36 bp, Artificial Sequence; identical to SEQ ID NO: 10
  Synthetic oligonucleotide primer

SEQ ID NO: 12 - DNA, 36 bp, Artificial Sequence
  Synthetic oligonucleotide primer
acacggccgtgtattactgtgcgagagaacgttcag

(19) United States
(12) Patent Application Publication
(10) Pub. No.: US 2009/0209432 A1
(43) Pub. Date: Aug. 20, 2009

(54) Detecting targets using nucleic acids having both a variable region and a conserved region

(75) Inventors: Alexander Alan Morley, South Australia (AU); Michael Julian Brisco, South Australia (AU); Pamela Joy Sykes, South Australia (AU)

Correspondence Address: SCULLY SCOTT MURPHY & PRESSER, PC, 400 GARDEN CITY PLAZA, SUITE 300, GARDEN CITY, NY, 11530, US

(73) Assignee: FLINDERS TECHNOLOGIES PTY. LTD., Bedford Park, South Autralia (AU)

(21) Appl. No.: 11/578,006

(22) PCT Filed: Apr. 6, 2005
(86) PCT No.: PCT/AU2005/000498

§ 371 Date: Dec. 5, 2008

(51) Int. Cl. C40B 30/04; C40B 40/06; C07H 21/00; C12Q 1/68; C40B 40/08

(52) U.S. Cl. 506/9; 506/16; 536/23.1; 506/17; 536/24.33; 435/6

(57) ABSTRACT

The invention relates to nucleic acid molecules for use in detecting a target nucleic acid molecule which is a member of a class of nucleic acid molecules and which is characterised by a specific variant region, said nucleic acid molecule comprising (i) a nucleic acid stem region which comprises a nucleic acid interaction site directed to a conserved region of the class of which said target nucleic acid molecule is member, or part thereof and which conserved region is located proximally to a variant region; operate y linked to (ii) a nucleic acid recognition region comprising at least two nucleotides. The nucleic acids are used in arrays and are an efficient means of screening molecules exhibiting a unique nucleotide sequence within a randomly varying population. The invention is useful in monitoring the effectiveness of therapeutic drug therapies and the progression of medical conditions, characterised by the presence of clonal populations of cells, particularly clonal lymphocyte populations.

### FIELD OF THE INVENTION

The present invention is directed to a novel array of oligonucleotides and methods for use thereof. More particularly, the present invention is directed to a novel array of amplification primers which facilitate the amplification of a specific nucleotide molecule of interest from a population of molecules, which vary randomly in sequence from one molecule to the other, in an efficient manner. The design of this array has now facilitated the development and implementation of very efficient means of screening for molecules exhibiting a unique nucleotide sequences within a randomly varying population. Accordingly, the molecules of the present invention are useful in a range of applications including, but not limited to, monitoring the progression of a condition characterised by the presence of a clonal population of cells, in particular a clonal lymphocyte population, monitoring the levels of a clonal cell population, predicting the likelihood of a subject's relapse from a remissive state to a disease state or for assessing the effectiveness of existing therapeutic drugs and/or new therapeutic drugs.

### BACKGROUND OF THE INVENTION

The reference to any prior art in this specification is not, and should not be taken as, an acknowledgment or any form of suggestion that that prior art forms part of the common general knowledge in Australia.

Lymphocytes, the cells that subserve the immune response, are of two types—B lymphocytes which produce antibody and T lymphocytes which are involved in cellular immunity. During development, in order to develop a specific immune response, each lymphocyte rearranges in a unique fashion one or a few specific genes—the immunoglobulin genes for a B lymphocyte and the T cell receptor genes for a T lymphocyte. All descendants of these lymphocytes will then carry the same rearrangement.

A neoplasm is believed to arise as the result of summation of genetic changes in a single cell. Subsequent proliferation of that cell gives rise to a population of descendants. Neoplasms arising from malignant change in a B or T lymphocyte (often referred to as “cancers”) would therefore predominantly comprise a clone of cells, each of which contains the same gene rearrangements that were present in the founder cell, although secondary gene rearrangements may occur within the neoplastic clone leading to genetically different subclones.

Lymphocytic neoplasms are therefore clonal disorders. They develop after one or more mutations in a single cell cause the cell and its progeny to multiply progressively and exponentially. For example, when lymphocytic leukemic clones number 10¹¹ to 10¹² cells in the body, clinical symptoms ensue. Without treatment, the clone continues to expand, and death results when there are approximately 10¹³ leukemic cells. If, however, the patient receives cytotoxic treatment, the clone decreases in size, and it can no longer be identified by conventional techniques when it comprises fewer than about 10¹⁰ cells. At this point, the patient is judged to be in clinical and haematological remission, although the term “remission”, in fact, refers only to a somewhat arbitrary point toward one end of a continuum of leukemic-cell number. Since the number of leukemic cells that may remain during remission is unknown and may range from 0 to 10¹⁰, treatment after remission has been achieved is empirical and its intensity is based on various clinical or laboratory prognostic factors determined at diagnosis or early in treatment. Consequently, some patients may receive too little treatment and others may receive too much.

Current methods for monitoring malignant lymphocytes involve the use of a “marker” which is shared by all cells of the clone. The marker may be a surface antigen, or patterns of several surface antigens, or it may be a molecular change. The molecular changes which are used may be broadly classified into two types—those which involve a chromosomal translocation or inversion, and those which will use the immunoglobulin or T cell receptor (herein referred to as “TCR”) gene arrangements.

In the context of screening for immunoglobulin or TCR gene rearrangements, as a means of analysing a particular clonal cell population, variability exists in the nucleotide sequences of the rearranged variable region of a population of B cells or T cells. On occasions the variability may involve one or only a few bases, when for example polymorphisms exist in the population. Under such circumstances it may be feasible to amplify from these slightly variable regions by having a small panel, usually one or several, PCR primers, which are usually constructed to bind to the range of known variants. However, in many situations the variability is too great and, in amplifying a particular sample, the conventional approach is therefore to sequence the region of interest and synthesise a specific primer or primers which binds to the particular sequence of the region of interest. Until the advent of the present invention, however, it has not proved practical to pre-synthesise primer arrays for ongoing use in amplifying nucleic acid molecules of interest from a class of molecules exhibiting regions of extensive variability, owing to the number of primers which would be required. For example, in considering a region of n nucleotides, each of which may be adenine, guanine, cytosine or thymine, the number of possible combinations is 4ⁿ.

Although the technology for synthesising a specific primer is widely available, the time and cost involved in doing so can become prohibitive where one is required to use many different primer sequences, for example, where a clinical or diagnostic laboratory requires access to a unique primer for each individual patient who is under investigation. As detailed above, to pre-synthesise an array of primers, for repeated use as a primer source, from which a suitable primer could be selected for the amplification of a region of high variability, the large number of primers which would be required to be synthesised is prohibitive both in terms of cost and practicality. Accordingly, there is a need to develop means of facilitating the efficient and routine amplification of a specific marker sequence for any given patient.

In work leading up to the present invention, the inventors have developed a means of designing a panel of pre-synthesised oligonucleotide primers from which one can select an appropriate oligonucleotide primer for amplifying a specific sequence of DNA, such as a neoplastic lymphoid cell rearranged immunoglobulin or TCR gene region, which randomly varies from one cell population to the next.

The invention has been developed in light of the determination that a target nucleic acid molecule comprising the feature that a variant nucleotide region (as between members of the class to which it belongs) is positioned adjacent to a substantially invariant (conserved) nucleotide region can be selected for by primers which are specifically designed to exploit this feature. The nature of the design of this array of oligonucleotides can facilitate the amplification of a target nucleotide sequence, even where a very high degree of nucleotide variation exists across different members of the class to which it belongs, without the need to synthesise a prohibitively large panel of oligonucleotides for ongoing use. In one example, the primers comprising an array are designed with a 5′ stem region, which recognises the substantially conserved region of the target nucleic acid molecule abutting the variant region, and a 3′ recognition end which comprises a random combination of two or more nucleotides, and which will interact with a complementary variant region. Preferably, the array comprises primers which correspond to each of the possible 16, 64 or 256 combinations of nucleotides for the 3′ recognition end, where that 3′ end consists of 2, 3 or 4 nucleotides, respectively for example, if the 3′ recognition end comprises more than 4 nucleotides, then the number of primers constituting the array will increase. By selecting and utilising an appropriately designed oligonucleotide from the array, either in isolation or in tandem with another suitable oligonucleotide, as hereinafter explained in more detail, any target nucleic acid sequence variant of interest can ultimately be selectively amplified. The development of these oligonucleotide arrays now facilitates the development of cost effective, rapid and routine means of screening for any one or more specific target nucleic acid molecules within a class of nucleic acid molecules which are defined by a substantially conserved nucleotide sequence region positioned adjacent to a variable sequence region.

### SUMMARY OF THE INVENTION

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

The subject specification contains nucleotide sequence information prepared using the programme PatentIn Version 3.1, presented herein after the bibliography. Each nucleotide sequence is identified in the sequence listing by the numeric indicator <210> followed by the sequence identifier (eg. <210>1, <210>2, etc). The length, type of sequence (DNA, etc) and source organism for each nucleotide sequence is indicated by information provided in the numeric indicator fields <211>, <212> and <213>, respectively. Nucleotide sequences referred to in the specification are identified by the indicator SEQ ID NO: followed by the sequence identifier (eg. SEQ ID NO:1, SEQ ID NO:2, etc.). The sequence identifier referred to in the specification correlates to the information provided in numeric indicator field <400> in the sequence listing, which is followed by the sequence identifier (eg. <400>1, <400>2, etc). That is SEQ ID NO: 1 as detailed in the specification correlates to the sequence indicated as <400>1 in the sequence listing.

One aspect of the present invention is directed to an array of isolated nucleic acid molecules or derivatives or analogues thereof, for use in detecting a target nucleic acid molecule which is a member of a class of nucleic acid molecules and which is characterised by a specific variant region said nucleic acid molecules comprising:
(i) a nucleic acid stem region, which stem region comprises a nucleic acid interaction site directed to a substantially conserved region of the class of which said target nucleic acid molecule is a member, or part thereof and which substantially conserved region is located proximally to a variant region operably linked to
ii) a nucleic acid recognition region comprising at least two nucleotides
   wherein said nucleic acid molecules comprise unique nucleic acid recognition region sequences relative to one another and wherein said nucleic acid molecules optionally comprise one or more universally hybridising bases, or analogues thereof, intervening said stem region and said recognition region.

Another aspect of the present invention is directed to an array of isolated oligonucleotides or derivatives or analogues thereof, for use in detecting a target nucleic acid molecule which is a member of a class of nucleic acid molecules and which is characterised by a specific variant region, said oligonucleotides comprising:
(i) a nucleic acid stem region, which stem region comprises a nucleic acid interaction site directed to a substantially conserved region of the class of which said target nucleic acid molecule is a member, or part thereof and which substantially conserved region is located proximally to a variant region; operably linked to
(ii) a nucleic acid recognition region comprising at least two nucleotides
   wherein said oligonucleotides comprise unique nucleic acid recognition region sequences relative to one another and wherein said oligonucleotides optionally comprise one or more universally hybridising bases, or analogues thereof, intervening said stem region and said recognition region.

Yet another aspect of the present invention is directed to an array of isolated oligonucleotide primers or derivatives or analogues thereof for use in detecting a target nucleic acid molecule which is a member of a class of nucleic acid molecules and which is characterised by a specific variant region, said oligonucleotide primers comprising:
(i) a nucleic acid stem region, which stem region comprises a nucleic acid interaction site directed to a substantially conserved region of the class of which said target nucleic acid molecule is a member, or part thereof, and which substantially conserved region is located proximally to a variant region; operably linked to
(ii) a nucleic acid recognition region comprising at least two nucleotides
   wherein said oligonucleotide primers comprise unique nucleic acid recognition region sequences relative to one another and wherein said oligonucleotides optionally comprise one or more universally hybridising bases, or analogues thereof, intervening said stem region and said recognition region.

Still another aspect of the present invention provides an array of isolated DNA primers or derivatives or analogues thereof, for use in detecting a target gene which is a member of a class of genes which are characterised by a specific variant region said primers comprising:
(i) a nucleic acid stem region, which stem region comprises a nucleic acid interaction site directed to a substantially conserved region of the class of which said target gene, or part thereof, is a member, which substantially conserved region is located proximally to said variant region; operably linked to
(ii) a nucleic acid recognition region comprising at least two nucleotides
   wherein said primers comprise unique nucleic acid recognition region sequences relative to one another and wherein said primers optionally comprise one or more universally hybridising bases, or analogues thereof, intervening said stem region and said recognition region.

Yet still another aspect of the present invention is directed to an array of isolated DNA primers or derivatives or analogues thereof for use in detecting a rearranged TCR or immunoglobulin variable gene segment, or part thereof, said primers comprising:
(i) a nucleic acid stem region, which stem region comprises a nucleic acid interaction site directed to a substantially conserved region of the TCR or immunoglobulin variable gene segment and which substantially conserved region is located proximally to a variant region; operably linked to
(ii) a nucleic acid recognition region comprising at least two nucleotides
   wherein said primers comprise unique nucleic acid recognition region sequences relative to one another and wherein said primers optionally comprise one or more universally hybridising bases, or analogues thereof, intervening said stem region and said recognition region.

Still yet another aspect of the present invention is directed to an array of isolated DNA primers or derivatives or analogues thereof for use in detecting a rearranged TCR or immunoglobulin variable gene segment, or part thereof, said primers comprising:
(i) a nucleic acid stem region, which stem region comprises a nucleic acid interaction site directed to a substantially conserved portion of the 5′ end of the antisense strand of the V gene segment, or part thereof, operably linked to
(ii) a nucleic acid recognition region comprising at least two nucleotides
   wherein said primers comprise unique nucleic acid recognition region sequences relative to one another and wherein said primers optionally comprise one or more universally hybridising bases, or analogues thereof, intervening said stem region and said recognition region.

A further aspect of the present invention is directed to an array of isolated DNA primers or derivatives or analogues thereof for use in detecting a rearranged TCR or immunoglobulin variable gene segment, or part thereof, said primers comprising:
(i) a nucleic acid stem region, which stem region comprises a nucleic acid interaction site directed to a substantially conserved portion of the 5′ end of the antisense strand of the D gene segment, or part thereof, operably linked to
(ii) a nucleic acid recognition region comprising at least two nucleotides
   wherein said primers comprise unique nucleic acid recognition region sequences relative to one another and wherein said primers optionally comprise one or more universally hybridising bases, or analogues thereof, intervening said stem region and said recognition region.

In yet another preferred embodiment of the present invention is directed to an array of isolated DNA primers or derivatives or analogues thereof for use in detecting a rearranged TCR or immunoglobulin variable gene segment, or part thereof, said primers comprising:
(i) a nucleic acid stem region, which stem region comprises a nucleic acid interaction site directed to a substantially conserved region of the 5′ end of the sense strand of the J gene segment, or part thereof; operably linked to
(ii) a nucleic acid recognition region comprising at least two nucleotides
   wherein said primers comprise unique nucleic acid recognition region sequences relative to one another and wherein said primers optionally comprise one or more universally hybridising bases, or analogues thereof, intervening said stem region and said recognition region.

Another further aspect of the present invention provides an array of isolated DNA primers or derivatives or analogues thereof, for use in detecting a rearranged TCR or immunoglobulin variable gene segment, or part thereof, said primers comprising:
(i) a nucleic acid stem region, which stem region comprises a nucleic acid interaction site directed to the 5′ end of the antisense strand of the V gene segment, or part thereof; operably linked to
(ii) a nucleic acid recognition region comprising at least two nucleotides
   wherein said primers comprise unique nucleic acid recognition region sequences relative to one another and wherein said primers optionally comprise one or more inosines, or analogues thereof, intervening said stem region and said recognition region.

In another aspect there is provided an array of isolated DNA primers or derivatives or analogues thereof, for use in detecting a rearranged TCR or immunoglobulin variable gene segment, or part thereof, said primers comprising:
(i) a nucleic acid stem region, which stem region comprises a nucleic acid interaction site directed to the 5′ end of the antisense strand of the D gene segment, or part thereof; operably linked to
(ii) a nucleic acid recognition region comprising at least two nucleotides wherein said primers comprise unique nucleic acid recognition region sequences relative to one another and wherein said primers optionally comprise one or more inosines, or analogues thereof, intervening said stem region and said recognition region.

In still another preferred embodiment there is provided an array of isolated DNA primers or derivatives or analogues thereof, for use in detecting a rearranged TCR or immunoglobulin variable gene segment, or part thereof, said primers comprising:
(i) a nucleic acid stem region, which stem region comprises a nucleic acid interaction site directed to the 5′ end of the sense strand of the J gene segment, or part thereof; operably linked to
(ii) a nucleic acid recognition region comprising at least two nucleotides
   wherein said primers comprise unique nucleic acid recognition region sequences relative to one another and wherein said primers optionally comprise one or more inosines, or analogues thereof, intervening said stem region and said recognition region.

In yet another further aspect the present invention provides an array of isolated DNA primers or derivatives or analogues thereof, for use in detecting a target nucleic acid molecule which is a member of a class of nucleic acid molecules which are characterised by a specific variant region sequence, said primers comprising:
(i) a nucleic acid stem region, which stem region comprises a nucleic acid interaction site directed to a substantially conserved region of the class of which said target nucleic acid molecule is a member, or part thereof, and which substantially conserved region is located proximally to a variant region; operably linked to
(ii) a nucleic acid recognition region comprising three nucleotides
   wherein said primers comprise unique nucleic acid recognition region sequences relative to one another and wherein said primers optionally comprise at least two inosines, or analogues thereof, intervening said stem region and said recognition region.

In still another further aspect there is provided an array of isolated DNA primers or derivatives or analogues thereof, for use in detecting a target nucleic acid molecule which is a member of a class of nucleic acid molecules which are characterised by a specific variant region sequence, said primers comprising:
(i) a nucleic acid stem region, which stem region comprises a nucleic acid interaction site directed to a substantially conserved regions of the class of which said target nucleic acid molecule is a member, or part thereof, and which substantially conserved region is located proximally to a variant region; operably linked to
(ii) a nucleic acid recognition region comprising four nucleotides
   wherein said primers comprise unique nucleic acid recognition region sequences relative to one another and wherein said primers optionally comprise at least two inosines, or analogues thereof, intervening said stem region and said recognition region.

Another aspect of the present invention is directed to a method of identifying a target nucleic acid molecule in a sample, which molecule is a member of a class of nucleic acid molecules characterised by a specific variant region sequence, said method comprising
(i) contacting said sample with an oligonucleotide as hereinbefore defined for a time and under conditions sufficient to facilitate interaction of said oligonucleotide with said target nucleic acid molecule;
(ii) amplifying said nucleic acid target; and
(iii) optionally consecutively repeating said amplification steps utilising the nucleic acid material amplified in the preceding step together with a leap frog oligonucleotide; and
(iv) detecting said amplified product.

Another aspect of the present invention provides a method of detecting and/or monitoring a clonal population of cells in a mammal, which clonal cells are characterised by a target nucleic acid molecule which is a member of a class of nucleic acid molecules characterised by a specific variant region sequence, said method comprising:
(i) contacting the nucleic acid material of a biological sample derived from a mammal with an oligonucleotide as hereinbefore defined for a time and under conditions sufficient to facilitate interaction of said oligonucleotide with said target nucleic acid molecule;
(ii) amplifying said nucleic acid target;
(iii) optionally consecutively repeating said amplification steps utilising the nucleic acid material amplified in the preceding step together with a leap frog oligonucleotide; and
(iv) detecting said amplified product.

Accordingly, still another aspect of the present invention is directed to a method for diagnosis of the onset of or a predisposition to the onset of a disease condition or for monitoring or prognosing the progression of a disease condition in a mammal, which condition is characterised by the presence or change in the level of a target nucleic acid molecule, or clonal cell population characterised by a target nucleic acid molecule, which molecule is a member of a class nucleic acid molecule characterised by a specific variant region sequence, said method comprising:
(i) contacting a sample derived from said mammal with an oligonucleotide as hereinbefore defined, for a time and under conditions sufficient to facilitate interaction of said oligonucleotide with said target nucleic acid molecule;
(ii) amplifying said nucleic acid target;
(iii) optionally consecutively repeating said amplification steps utilising the nucleic acid material amplified in the preceding step together with a leap frog oligonucleotide; and
(iv) detecting said amplified product.

Yet another aspect of the present invention is directed to a kit for facilitating the identification of a target nucleic acid molecule, said kit comprising compartments adapted to contain any one or more of the oligonucleotide primers as hereinbefore defined, reagents useful for facilitating interaction of said primer with the target nucleic acid molecule and reagents useful for enabling said interaction to result in amplification of said nucleic acid target. Further compartments may also be included, for example, to receive biological or non-biological samples.

### BRIEF DESCRIPTION OF THE DRAWINGS

FIG. 1 is a graphical representation of the effect of the number of inosines and the temperature of annealing on the degree of amplification achieved by 20 cycles of PCR.

FIG. 2 is a graphical representation of the observed vs expected minimal residual disease (“MRDI”).

FIG. 3 is a schematic representation of an example of a primary primer and leap frog primer used to amplify the desired nucleotide sequence in a specific fashion. The primary primer and the leap frog primer are each chosen from arrays, which in this case, each comprise 64 (4⁴) members. The stem region (solid line) and the intervening region (dashed line), which shows some variability, are shown, together with the next 8 bases of all possible sequences. The bases of the sequence which it is designed to amplify are shown in bold and the sequence recognised is ACGTTCAG. The nucleic acid molecules which it is desired to detect are those which contain this sequence.

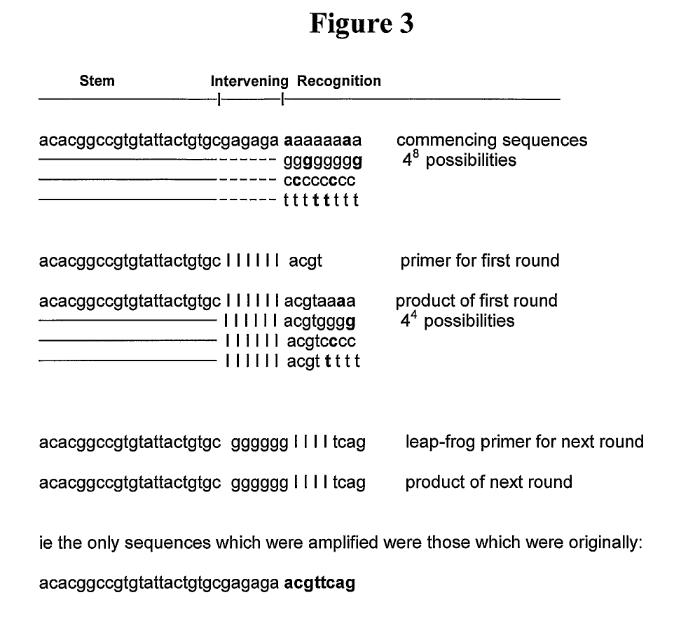

FIG. 4 is a graphical representation of the paired comparisons of performance of 10 inosine primers with 10 standard primers. For each pair the standard primer had exactly the same sequence as the inosine primer except that the 6 inosines were replaced by the 6 bases actually present in the particular gene rearrangement. Rearrangements from 10 leukaemic marrow samples at diagnosis (closed symbols) and from 10 peripheral blood samples from normal individuals (open symbols) were studied by quantitative PCR with the end-point of cycles to threshold (Ct). The inosine primers showed essentially the same Ct values as the standard primers, indicating that they amplified with the same efficiency. The Cts for the monoclonal leukaemic samples were 6-12 cycles less than for the polyclonal peripheral blood samples. This difference indicates good specificity and is of the order of difference expected, as all of the rearrangements in leukemic DNA would be expected to amplify whereas approximately only 1 in 4⁴ ie, 1 in 256, of the rearrangements in peripheral blood DNA would be expected to do so.

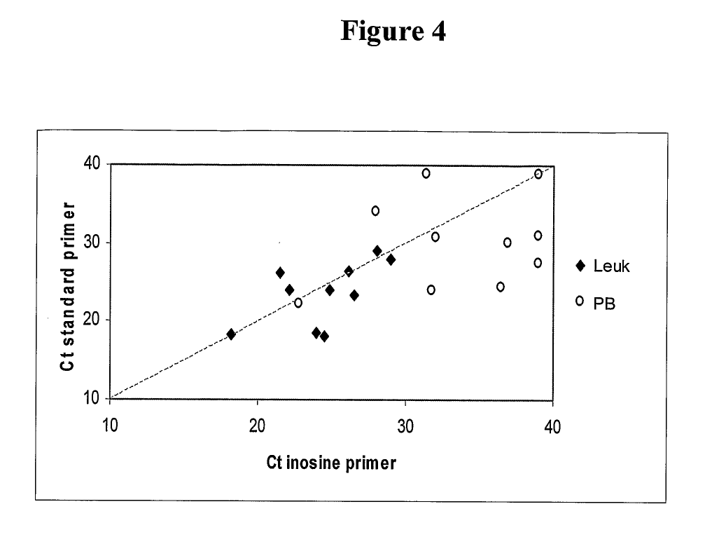

FIG. 5 is a graphical representation of the results of measurement of minimal residual disease (MRD, the proportion of leukaemic cells) in the marrow in 8 adults with acute lymphoblastic leukaemia. The patients were participating in a trial carried out by the Australasian Leukaemia lymphoma Group which was investigating the utility of a new form of drug treatment. Bone marrows were done at diagnosis and on days 28 and 56 of treatment. MRD estimations on days 28 and 56 were performed using 3 rounds of PCR, the last round being quantitative real-time PCR. The first round used V and J primers specific for the IgH rearrangement involved, the second round used primers containing 6 inosines and terminating at the 3′ end with the 4 patient-specific nucleotides, and the third round used either a D-region specific primer, a specific leap-frog inosine-containing primer, or a patient-specific primer. When an inosine primer was used, annealing was performed at 43 deg.C. Experiments have shown that good specificity and sensitivity is obtained at this temperature.

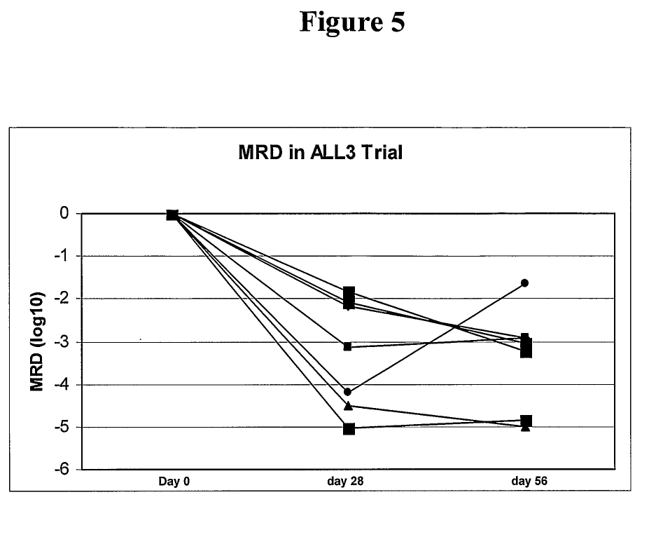

### DETAILED DESCRIPTION OF THE INVENTION

The present invention is predicated, in part, on the determination that one can design an array of pre-synthesised oligonucleotide primers from which can be selected a primer capable of amplifying any one of the members of a class of nucleic acid molecules which exhibit a region of nucleotide sequence variation adjacent to a region of substantial conservation of sequence (such as occurs in the context of rearranged TCR or immunoglobulin genes). Importantly, this can be efficiently effected without the need to necessarily generate the prohibitively large numbers of distinct primers which, to date, would have been required to provide an effective means of achieving this type of target nucleic acid selection. Specifically, due to the design of primers which exploit the region of the target sequence where the substantially invariant/conserved sequence abuts the variant sequence, a suitable primer can be selected from an array of as few as 4²-4⁴ oligonucleotides to achieve highly specific selection either via a single amplification step or by consecutive amplification steps which use primers appropriately selected from two or more distinct arrays. This system of consecutive amplification facilitates a high level of sensitivity and is hereinafter described in more detail. The development of these arrays has now facilitated their application to detecting or monitoring conditions characterised by the presence of cells, in particular clonal populations of cells, expressing a nucleic acid molecule exhibiting this type of structure.

Accordingly, one aspect of the present invention is directed to an array of isolated nucleic acid molecules or derivatives or analogues thereof, for use in detecting a target nucleic acid molecule which is a member of a class of nucleic acid molecules and which is characterised by a specific variant region said nucleic acid molecules comprising:
i) a nucleic acid stem region, which stem region comprises a nucleic acid interaction site directed to a substantially conserved region of the class of which said target nucleic acid molecule is a member, or part thereof and which substantially conserved region is located proximally to a variant region operably linked to
ii) a nucleic acid recognition region comprising at least two nucleotides
   wherein said nucleic acid molecules comprise unique nucleic acid recognition region sequences relative to one another and wherein said nucleic acid molecules optionally comprise one or more universally hybridising bases, or analogues thereof, intervening said stem region and said recognition region.

Reference to a “nucleic acid molecule” for use in “detecting a target” should be understood as a reference to a nucleic acid molecule or derivative or analogue thereof which functions to identify, isolate and/or enrich a target molecule. Examples of such molecules include, but are not limited to, nucleic acid molecules which can function as probes and/or amplification primers. Preferably, the subject nucleic acid molecule is an oligonucleotide of 4 to 60 nucleotides in length, preferably 10 to 50 in length, more preferably 15 to 45 in length, still more preferably 20 to 40 in length, yet more preferably 25 to 35 in length. Most preferably, said nucleic acid molecule is about 26, 27, 28, 29, 30, 31, 32, 33 or 34 nucleotides in length.

More particularly, the present invention is directed to an array of isolated oligonucleotides or derivatives or analogues thereof, for use in detecting a target nucleic acid molecule which is a member of a class of nucleic acid molecules and which is characterised by a specific variant region, said oligonucleotides comprising:
(i) a nucleic acid stem region, which stem region comprises a nucleic acid interaction site directed to a substantially conserved region of the class of which said target nucleic acid molecule is a member, or part thereof and which substantially conserved region is located proximally to a variant region; operably linked to
(ii) a nucleic acid recognition region comprising at least two nucleotides
   wherein said oligonucleotides comprise unique nucleic acid recognition region sequences relative to one another and wherein said oligonucleotides optionally comprise one or more universally hybridising bases, or analogues thereof, intervening said stem region and said recognition region.

Preferably, said oligonucleotide is an oligonucleotide primer.

Accordingly, still more particularly, the present invention is directed to an array of isolated oligonucleotide primers or derivatives or analogues thereof for use in detecting a target nucleic acid molecule which is a member of a class of nucleic acid molecules and which is characterised by a specific variant region, said oligonucleotide primers comprising:
(i) a nucleic acid stem region, which stem region comprises a nucleic acid interaction site directed to a substantially conserved region of the class of which said target nucleic acid molecule is a member, or part thereof, and which substantially conserved region is located proximally to a variant region; operably linked to
(ii) a nucleic acid recognition region comprising at least two nucleotides
   wherein said oligonucleotide primers comprise unique nucleic acid recognition region sequences relative to one another and wherein said oligonucleotides optionally comprise one or more universally hybridising bases, or analogues thereof, intervening said stem region and said recognition region.

Reference to a “nucleic acid” or “nucleotide” should be understood as a reference to both deoxyribonucleic acid or nucleotides and ribonucleic acid or nucleotides or derivatives or analogues thereof. In this regard, it should be understood to encompass phosphate esters of ribonucleotides and/or deoxyribonucleotides, including DNA (cDNA or genomic DNA), RNA, mRNA or tRNA among others. The nucleic acid molecules of the present invention may be of any origin including naturally occurring (such as would be derived from a biological sample), recombinantly produced or synthetically produced.

Reference to “derivatives” should be understood to include reference to fragments, parts, portions, homologs and mimetics of said nucleic acid molecules from natural, synthetic or recombinant sources. “Functional derivatives” should be understood as derivatives which exhibit any one or more of the functional activities of nucleotides or nucleic acid molecules. The derivatives of said nucleotides or nucleic acid sequences include fragments having particular regions of the nucleotide or nucleic acid molecule fused to other proteinaceous or non-proteinaceous molecules. “Analogs” contemplated herein include, but are not limited to, modifications to the nucleotide or nucleic acid molecule such as modifications to its chemical makeup or overall conformation. This includes, for example, modification to the manner in which nucleotides or nucleic acid molecules interact with other nucleotides or nucleic acid molecules such as at the level of backbone formation or complementary base pair hybridisation. The biotinylation of a nucleotide or nucleic acid molecules is an example of a “functional derivative” as herein defined. Derivatives of nucleic acid molecules may be derived from single or multiple nucleotide substitutions, deletions and/or additions. The term “functional derivatives” should also be understood to encompass nucleotides or nucleic acid exhibiting any one or more of the functional activities of a nucleotide or nucleic acid sequence, such as for example, products obtained following natural product screening.

Reference to an “oligonucleotide primer” or “primer” should be understood as a reference to any molecule comprising a sequence of nucleotides, or functional derivatives or analogues thereof, the function of which includes the hybridisation of at least one region of said nucleotide sequence with a target nucleic acid molecule and the amplification of said target sequence. Accordingly, reference to a “target nucleic acid molecule” is a reference to any molecule comprising a sequence of nucleotides or functional derivative or analogue thereof which molecule is a molecule of interest and is therefore the subject of identification via an amplification step. Preferably, the target nucleic acid molecule is a gene, or part thereof, such as one or more of the junction regions of the rearranged V, D or J segments of the TCR or immunoglobulin genes.

Both the primer and the target nucleic acid molecule may comprise non-nucleic acid components. For example, the primer may also comprise a non-nucleic acid detection tag (for example, allowing it to additionally or alternatively function as a probe), such as a fluorescent tag or some other non-nucleic acid component which facilitates the functioning of the molecule, such as the detection or immobilisation of the molecule. Similarly, the target nucleic acid molecule may comprise a non-nucleic acid component. For example, the target nucleic acid molecule may be bound to an antibody. This may occur, for example, where the target nucleic acid molecule is present in a biological sample isolated from an individual who is mounting an immune response, such as an autoimmune response, to said target nucleic acid molecule. In another example, the primer may be a protein nucleic acid which comprises a peptide backbone exhibiting nucleic acid side chains. Preferably, said target nucleic acid molecules is a gene region and said oligonucleotide primer is a DNA primer.

The present invention therefore preferably provides an array of isolated DNA primers or derivatives or analogues thereof, for use in detecting a target gene which is a member of a class of genes which are characterised by a specific variant region said primers comprising:
(i) a nucleic acid stem region, which stem region comprises a nucleic acid interaction site directed to a substantially conserved region of the class of which said target gene, or part thereof, is a member, which substantially conserved region is located proximally to said variant region; operably linked to
(ii) a nucleic acid recognition region comprising at least two nucleotides
   wherein said primers comprise unique nucleic acid recognition region sequences relative to one another and wherein said primers optionally comprise one or more universally hybridising bases, or analogues thereof, intervening said stem region and said recognition region.

It should be understood that the phrase “characterised by” is intended to indicate that the subject target nucleic acid molecule exhibits the defined characteristic but it is not intended as a limitation in respect of what other characteristics the molecule might also exhibit. It should also be understood that the subject characteristic is not necessarily uniquely exhibited only by the subject target molecule although in a preferred embodiment the characteristic is one which identifies the molecule of interest from the molecules of non-interest which are present in a sample.

The present invention is predicated on the finding that some classes of target nucleic acid molecules (for example, certain classes of genes) are characterised by a nucleotide sequence which comprises a substantially conserved region of sequence which is located proximally to a region which exhibits some degree of variation of sequence from one molecule to another. This may occur, for example, in genes which exhibit polymorphic variations. In another example, genes which undergo rearrangements, such as the TCR and immunoglobulin genes fall into this class. In yet another example, the target variation may be the result of non-natural gene mutation events such as recombinant engineering of a gene or random mutation due to toxic environmental factors. In this regard, reference to a “class” of molecules should be understood as a reference to a group of nucleic acid molecules, preferably genes, which exhibit a level of sequence homology high enough that they can be characterised as members of a single class of molecules (such as a single class of gene) but which members nevertheless exhibit unique differences in regions of their actual nucleic acid sequences. Preferably, the subject sequences are of the class of rearranged or at least partly rearranged immunoglobulin or TCR variable receptor gene families.

Accordingly, in a preferred embodiment, the present invention is directed to an array of isolated DNA primers or derivatives or analogues thereof for use in detecting a rearranged TCR or immunoglobulin variable region gene, or part thereof, said primers comprising:
(i) a nucleic acid stem region, which stem region comprises a nucleic acid interaction site directed to a substantially conserved region of the TCR or immunoglobulin variable region gene and which substantially conserved region is located proximally to a variant region; operably linked to
(ii) a nucleic acid recognition region comprising at least two nucleotides
   wherein said primers comprise unique nucleic acid recognition region sequences relative to one another and wherein said primers optionally comprise one or more universally hybridising bases, or analogues thereof, intervening said stem region and said recognition region.

Reference to a “substantially conserved region” should be understood as a reference to a region located proximally to the variant region and which is characterised by sufficient consensus sequence between the members of the class in issue that a primer can be designed which will bind most, preferably all, the members of the subject class at the conserved region. The subject conserved region is located proximally to a region which exhibits some degree of sequence variation. Without limiting the present invention to any one theory or mode of action, it is the existence and detection of the region of variation which enables the identification of a unique molecule within the class of nucleic acid molecules. Reference to “variant region” should therefore be understood as a reference to a region of nucleotides which exhibit variation of sequence between members of the class to which they belong. The degree of variation which exists in the context of a given class of molecules may in itself show significant differences between different classes of molecules. For example, the variation may involve one or only a few nucleotides, such as occurs in the context of a gene or class of genes which are subject to polymorphic variations. In other situations, however, the variation may be great. Accordingly, it should be understood that the “classes” of molecules to which this invention is directed may take any one of a number of forms including that the class corresponds to a family of genes one or more of which are present in the genome of the cells of a single individual, a cluster of genes which are the result of gene rearrangement, one or more of the rearranged genes being present in the genomes of specific cell populations of an individual or a single gene which exists in various polymorphic forms one or more of which forms can be found either within the genome of one individual (such as the MHC genes) or between individuals. It should also be understood that the variation may occur either as the result of naturally occurring molecular events, such as gene rearrangement or somatic mutations or it may be artificially induced such as can occur with exposure to chemicals, radiation or molecular engineering, for example.

It should also be understood that the subject conserved region and variant region may be arranged in any orientation. That is, the variant region may be located 3′ to the conserved region on the sense strand of the gene of interest or the variant region may be located 5′ to the conserved region of the sense strand. When one considers the antisense strand of the gene of interest, these 5′/3′ directional positions are reversed. For example, in the context of the sense strand of the immunoglobulin gene, the conserved V gene segment and the 3′ end of the D gene segment are positioned 5′ to the region of variation which abuts both these segments. However, the J segment and the 5′ end of the D segment of the sense strand are positioned in the 3′ direction relative to the region of variation. With respect to the antisense strand, these 5′ and 3′ directional positions are reversed, as detailed below:

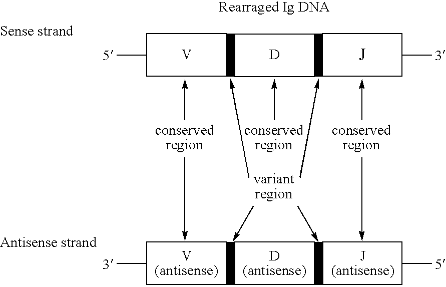

Accordingly, the particular strand of the double stranded target nucleic acid molecule to which the oligonucleotide primer of the present invention is directed will depend on the 5′/3′ orientation of the conserved and variant regions of the target nucleic acid molecule. In general, where one is seeking to detect a target molecule in which the variant region is located in the 3′ direction to the conserved region, such as occurs with the V segment of the immunoglobulin gene, it will be necessary to design a panel of primers which are directed to the antisense strand of this gene segment since this will facilitate extension of the primer from its 3′ end, that is from the terminal end of the recognition region of the primer. Accordingly, it should be understood that where the variant region of a target gene is located 5′ to the conserved region, such as occurs with the J segment of the immunoglobulin gene, the primer is designed such that it is directed to the sense strand of a target nucleic acid molecule.

Reference to the nucleic acid molecule which is detected using the primers of the present invention being “characterised by” a specific variant region should be understood as a reference to the sequence of the variant region being found in that nucleic acid molecule but which variant region is either not found in other nucleic acid molecules of that class or is not found at a significant level in other nucleic acid molecules. By “significant” is meant that the amplification of a population of nucleic acid molecules utilising a primer directed to that variant region nevertheless provides a useful indicator of the nucleic acid molecule of interest based either on a single step amplification reaction or consecutive reactions, as hereinafter described in more detail.

The primers of the present invention are directed to identifying a nucleic acid molecule which is characterised by a substantially conserved region located proximally to a variant region. By “proximally to” is meant that the regions are positioned relative to one another such that a primer can be designed to interact with both regions. Preferably, the variant region is immediately adjacent to the terminal end of the conserved region. However, it may also be located near to the conserved region and therefore separated by a number of intervening nucleotides.

In a preferred embodiment of the present invention, the nucleic acid molecule to which an array of primers is generated is the genomic rearranged variable region of the TCR or immunoglobulin genes. Without limiting the present invention in any way, each lymphoid cell undergoes somatic recombination of its germ line variable region gene segments (either V and J or V, D and J segments), depending on the particular gene arranged, in order to generate a total antigen diversity of approximately 10¹⁶ distinct variable region structures (note that the expression “variable gene segment” of the TCR or immunoglobulin gene is distinct from the expression “variant region” as hereinbefore defined). In any given lymphoid cell, such as a T cell or B cell, at least two distinct variable region gene segment rearrangements are likely to occur due to rearrangements involving the α, β, γ or δ chain genes of the TCR gene family and/or the heavy and light chains of the immunoglobulin gene family. In addition to rearrangements of the VJ or VDJ segment of any given immunoglobulin or TCR gene, nucleotides are randomly removed and/or inserted at the junction between the segments. This leads to the generation of enormous diversity.

This preferred embodiment is an example of the situation where the conserved region (being the V, D or J segments) and the variant region (being the region of randomly inserted and/or deleted bases between the V, D and J segments) may either be directly linked or, where the random base changes have extended into the V, D or J segments, the conserved portion of the V, D or J segments is linked to the variant region via those intervening bases which have randomly mutated at the terminal ends of the V, D or J segments.

Accordingly, in a preferred embodiment the present invention is directed to an array of isolated DNA primers or derivatives or analogues thereof for use in detecting a rearranged TCR or immunoglobulin variable gene segment, or part thereof, said primers comprising:
(i) a nucleic acid stem region, which stem region comprises a nucleic acid interaction site directed to a substantially conserved portion of the 5′ end of the antisense strand of the V gene segment, or part thereof, operably linked to
(ii) a nucleic acid recognition region comprising at least two nucleotides
   wherein said primers comprise unique nucleic acid recognition region sequences relative to one another and wherein said primers optionally comprise one or more universally hybridising bases, or analogues thereof, intervening said stem region and said recognition region.

Preferably, said conserved portion of the V gene segment is a conserved portion of the F_{R3I} or F_{R3II} segment.

In another preferred embodiment the present invention is directed to an array of isolated DNA primers or derivatives or analogues thereof for use in detecting a rearranged TCR or immunoglobulin variable gene segment, or part thereof, said primers comprising:
(i) a nucleic acid stem region, which stem region comprises a nucleic acid interaction site directed to a substantially conserved portion of the 5′ end of the antisense strand of the D gene segment, or part thereof, operably linked to
(ii) a nucleic acid recognition region comprising at least two nucleotides
   wherein said primers comprise unique nucleic acid recognition region sequences relative to one another and wherein said primers optionally comprise one or more universally hybridising bases, or analogues thereof, intervening said stem region and said recognition region.

Preferably, said recognition region is operably linked to the 3′ end of the nucleic acid stem region of said oligonucleotide primer.

In yet another preferred embodiment the present invention is directed to an array of isolated DNA primers or derivatives or analogues thereof for use in detecting a rearranged TCR or immunoglobulin variable gene segment or part thereof, said primers comprising:
(i) a nucleic acid stem region, which stem region comprises a nucleic acid interaction site directed to a substantially conserved portion of the 5′ end of the sense strand of the J gene segment, or part thereof; operably linked to
(ii) a nucleic acid recognition region comprising at least two nucleotides
   wherein said primers comprise unique nucleic acid recognition region sequences relative to one another and wherein said primers optionally comprise one or more universally hybridising bases, or analogues thereof, intervening said stem region and said recognition region.

Without limiting the present invention to any one theory or mode of action, in order to hybridise with the target nucleic acid molecules hereinbefore defined, the primer is designed with a stem region operably linked to a recognition region. This design exploits the unique feature of the subject target molecules being that they comprise a variant region sequence, which is sufficiently unique to act as a marker, located proximally to a region which is substantially conserved across the class of which the target is a member. These features have facilitated the development of primer arrays suitable for amplifying any specific member of such a class of molecules due to the fact that the stem region of the primer enables identification of ay molecule falling within the class of interest while the recognition region enables identification, via either a one step or multiple step amplification process, of a specific member within that class. As discussed in further detail hereafter, however, the pre-synthesised primer arrays which are designed in accordance with the teachings provided herein can provide a high degree of amplification specificity and are suitable for ongoing use as a primer source for amplification of any given target molecule of interest within a class of target nucleic acid molecules, without the need to synthesise the prohibitively large numbers of primers which are currently required in order to achieve the same outcome.

In this regard, reference to “stem region” should be understood as a reference to that portion of the subject primer which interacts with the terminal nucleotides adjoining the variant region, of the substantially conserved region of the target molecule. In a most preferred embodiment these nucleotides are located at the 3′ terminal end of the substantially conserved region. To the extent that the substantially conserved region directly adjoins the variant region, the stem region is designed to hybridise to sufficient of the nucleic acid sequence leading up to the point where the conserved region adjoins the variant region such that the stem region would hybridise with any member of that class of molecules. This may be achieved, for example, by designing the stem region to hybridise to the 3′ terminal four or more nucleotides, where all members of the class comprise an identical sequence at that region. However, if there is very slight variation in sequence at that region between some members, the stem region may be designed to hybridise to a consensus sequence. Designing a suitable stem region, in terms of the number and sequence of the desired nucleotides, would fall well within the abilities of the person of skill in the art when considered in light of the teachings provided herein. Preferably, said stem region is 5-50 nucleotides in length, more preferably, 10-40 nucleotides in length, and even more preferably, 15, 20, 21, 22, 23, 24, 25, 26, 27, 28, 29, 30, 31, 32, 33, 34 or 35 nucleotides in length.

That the stem region comprises a “nucleic acid interaction site” should be understood as a reference to that portion of the stem region which actually hybridises to the conserved portion of the target nucleic acid molecule Although it is preferable, and likely, that in order to enable synthesis of the smallest possible DNA primer the entirety of the nucleic acid component of the stem region of the primer will correspond to the nucleic acid interaction site, this may not always be the case. In some instances, it may be the case that only part of the stem region corresponds to the nucleic acid interaction site. This may occur, for example, where the stem region of the primer is not linear in shape, but takes the form of a loop or contains a 5′ tag.

As discussed hereinbefore, in some instances one or more of the terminal nucleotides adjoining the variant region, of the substantially conserved region may themselves have undergone some degree of mutation or show some other variation between members of the class. It should be understood that, in accordance with the description of the invention as provided herein, these nucleotides are not deemed to form part of either the substantially conserved region or the variant region but correspond to a region of “intervening” nucleotides. Similarly, to the extent that the substantially conserved region does not directly link to the variant region in that there are one or more unrelated nucleotides positioned between the two regions, these are also an example of intervening nucleotides which, by definition, form part of neither the conserved or the variant regions. In this regard, however, the design of the primers of the present invention contemplate this scenario in that the primers may be optionally designed and synthesised such that they comprise one or more universally hybridising nucleotides which are positioned such that they intervene the stem region and the recognition region of the primer. Once more, it is well within the skill of the person in the art to determine the existence and number of any intervening nucleotides in the context of a class of molecules to which a primer set is being designed and pre-synthesised. In a preferred embodiment, the number of universally hybridising bases which are built into the primer between the stem region and the recognition region will correspond to the number of intervening nucleotides.

Reference to “universally hybridising base” should be understood as a reference to a molecule which can hybridise with all of guanine, cytosine, thymine, uridine or adenine. It should be understood, however, that there may exist differences in the strength of the hybridisation of the universally hybridising base with each of these five nucleotides. Accordingly, to the extent that some degree of hybridisation can be effected, the “base” in issue falls within the scope of this definition. It should also be understood that the subject base may be any nucleic acid or non-nucleic acid molecule which can function in accordance with the definition provided above. For example, there are a number of well known chemical modifications which can be made to the various bases which result in universal binding. Examples of universal bases include hypoxanthine, 5-nitroindole, 3-nitropyrrole, acyclic sugar analogues of hypoxanthine (18) and 5-nitroindazole, phenyl C-ribonucleoside, (Nucleic Acids Research, 2001, Vol. 29, No. 12 2437-2447) One may also use functionally equivalent means such as primers which are fully redundant at the site in issue. Accordingly, primers which include complete or near complete redundancy for cytosine, guanine, adenine, thymine or uridine at that base location in the primer are envisaged. Preferably the subject base is a nucleotide and even more preferably inosine or derivative, or analogue thereof.

There is therefore preferably provided an array of isolated DNA primers or derivatives or analogues thereof, for use in detecting a rearranged TCR or immunoglobulin variable gene segment, or part thereof, said primers comprising:
(i) a nucleic acid stem region, which stem region comprises a nucleic acid interaction site directed to the 5′ end of the antisense strand of the V gene segment, or part thereof; operably linked to
(ii) a nucleic acid recognition region comprising at least two nucleotides
   wherein said primers comprise unique nucleic acid recognition region sequences relative to one another and wherein said primers optionally comprise one or more inosines, or analogues thereof, intervening said stem region and said recognition region.

Preferably, said conserved portion of the V gene segment is a conserved portion of the F_{R3I} or F_{RII} segment.

In another preferred embodiment there is provided an array of isolated DNA primers or derivatives or analogues thereof, for use in detecting a rearranged TCR or immunoglobulin variable gene segment, or part thereof, said primers comprising:
(i) a nucleic acid stem region, which stem region comprises a nucleic acid interaction site directed to the 5′ end of the antisense strand of the D gene segment, or part thereof; operably linked to
(ii) a nucleic acid recognition region comprising at least two nucleotides
   wherein said primers comprise unique nucleic acid recognition region sequences relative to one another and wherein said primers optionally comprise one or more inosines, or analogues thereof, intervening said stem region and said recognition region.

In still another preferred embodiment there is provided an array of isolated DNA primers or derivatives or analogues thereof, for use in detecting a rearranged TCR or immunoglobulin variable gene segment, or part thereof, said primers comprising:
(i) a nucleic acid stem region, which stem region comprises a nucleic acid interaction site directed to the 5′ end of the sense strand of the J gene segment, or part thereof, operably linked to
(ii) a nucleic acid recognition region comprising at least two nucleotides
   wherein said primers comprise unique nucleic acid recognition region sequences relative to one another and wherein said primers optionally comprise one or more inosines, or analogues thereof, intervening said stem region and said recognition region.

The stem region of the primers of the present invention is operably linked to a “nucleic acid recognition region”. By “nucleic acid recognition region” is meant that portion of the subject primer which can discriminate between the members of a class of target nucleic molecules by hybridising to the variant region of a subgroup of molecules within that class. Preferably, the “subgroup” of molecules corresponds to a single target molecule of interest. However, since the primer array of the present invention is predicated on reducing the number of primers which are required to be synthesised for inclusion in a pre-synthesised array by minimising the number of nucleotides which form part of the recognition region and therefore minimising the total number of nucleotide combinations (4ⁿ) which are required to be generated to form a complete primer set, it is possible that some individual primers may hybridise with more than one member of a class of interest. As described hereinafter, this outcome may necessitate the application of an additional and subsequent amplification step utilising a primer selected from a pre-synthesised array which is nevertheless designed in accordance with the teachings provided hereinbefore but which can function to further discriminate the multiple target class members which may have been amplified by an initial round of amplification. The nucleic acid recognition region may comprise any number of nucleotides. The most suitable number of nucleotides is that number which both provides an acceptable level of discrimination (either in the context of a single step or multiple consecutive step amplification process) but minimises the number of primers required to be pre-synthesised in order to prepare a complete array (this number being 4ⁿ where n is the number of nucleotides comprising the nucleic acid recognition region). This number can be routinely determined by those of skill in the art. Specifically, the number is determined by balancing the extra specificity resulting from an increased number of nucleotides, as against the extra cost involved in synthesis and use. Preferred numbers of nucleotides comprising the recognition region are 2, 3, 4, 5 or 6, more preferably 3 or 4 and most preferably 4.

As detailed hereinbefore, the primer array of the present invention provides a pre-synthesised array from which a primer can be selected which will, either in a single step amplification process or a consecutive multi-step amplification process, detect a specific target nucleic acid molecule of interest. The target molecule is characterised by a unique variant region, a portion of which (2, 3 or 4 nucleotides at the 5′ terminal end of the variant region, for example) is the target for the primer's nucleic acid recognition region. In order to facilitate the generation of a primer array which can be used to supply a suitable primer to enable detection of any member of the defined class, it is necessary that primers are synthesised which correspond to every possible combination of adenine, guanine, cytosine and thymine/uracil. Accordingly, the number of primers which any given array comprises will equate to 4ⁿ where n is the number of nucleotides comprising the nucleic acid recognition region, as follows:

| Nucleotide number of primer | Primer array size |
|---|---|
| recognition region | (number of primers) |
| 2 | 16 |
| 3 | 64 |
| 4 | 256 |
| 5 | 1024 |
| 6 | 4096 |

Arguably, the primer arrays of 1024 and 4096 could be regarded as prohibitively large. Accordingly, and as detailed before, the preferred number of nucleotides comprising the primer recognition sequence is 3 or 4, most preferably 4, thereby dictating an array size of 64 or 256 respectively. In this regard, reference to the primers comprising a “unique nucleic acid region recognition sequence relative to one another” should be understood to mean that each of the primers comprises a unique combination of the nucleotides adenine, guanine, cytosine and thymine/uracil. However, although it is preferable that the array be designed such that it comprises one primer corresponding to each of the possible nucleotide combinations (thereby envisaging arrays of 64 or 256 primers, for example) the present invention nevertheless extends to arrays which may encompass multiple copies of any one or more primers or which do not include a primer corresponding to one or more specific nucleotide combinations. This may occur, for example, where it is known that a given class of target molecules does not or cannot comprise a variant region corresponding to a particular sequence. In this case it would therefore not be necessary to synthesise primers comprising a nucleic acid recognition region which is complementary to and would therefore hybridise with these non-existent variant regions.

In accordance with these preferred embodiments the present invention provides an array of isolated DNA primers or derivatives or analogues thereof, for use in detecting a target nucleic acid molecule which is a member of a class of nucleic acid molecules which are characterised by a specific variant region sequence, said primers comprising:
(i) a nucleic acid stem region, which stem region comprises a nucleic acid interaction site directed to a substantially conserved region of the class of which said target nucleic acid molecule is a member, or part thereof, and which substantially conserved region is located proximally to a variant region; operably linked to
(ii) a nucleic acid recognition region comprising three nucleotides
   wherein said primers comprise unique nucleic acid recognition region sequences relative to one another and wherein said primers optionally comprise at least two inosines, or analogues thereof, intervening said stem region and said recognition region.

In another preferred embodiment there is provided an array of isolated DNA primers or derivatives or analogues thereof, for use in detecting a target nucleic acid molecule which is a member of a class of nucleic acid molecules which are characterised by a specific variant region sequence, said primers comprising:
(i) a nucleic acid stem region, which stem region comprises a nucleic acid interaction site directed to a substantially conserved regions of the class of which said target nucleic acid molecule is a member, or part thereof, and which substantially conserved region is located proximally to a variant region; operably linked to
(ii) a nucleic acid recognition region comprising four nucleotides
   wherein said primers comprise unique nucleic acid recognition region sequences relative to one another and wherein said primers optionally comprise at least two inosines, or analogues thereof, intervening said stem region and said recognition region.

Still more preferably, said target nucleic acid molecule is a rearranged TCR or immunoglobulin variable region gene, or part thereof, and said substantially conserved portion is the 3′ end of the V gene segment.

The nucleic acid recognition region of the present invention is directed to a portion of the variant region of the target nucleic acid molecule of interest. In this regard, that portion may correspond to any part of the variant region and does not necessarily correspond to one of the terminal ends of that region, although this would be, in the first instance, a preferred option. That is, in one preferred embodiment a primer is designed in which the nucleic acid interaction site of the stem region is directed to a stretch of nucleotides at the 3′ terminal end of the substantially conserved portion of the target while the nucleic acid molecule recognition region is designed to hybridise to a nucleotide stretch at the 5′ terminal end of the variant region of the target molecule. However, there is sometimes not an abrupt transition between the substantially conserved region and the variant region, for example as occurs in the context of TCR variable gene rearrangements where the 3′ terminal nucleotides of the V region can be randomly mutated during the rearrangement event. There may also be random nucleotides inserted between the conserved region and the variant region. In this case, the primer is designed to incorporate a corresponding number of universally hybridising molecules (for example, inosine) intervening the stem region and the recognition region. In this situation (or even where the conserved and variant regions directly adjoin), a given primer may nevertheless detect and amplify more than one member of a class of target molecules. This may evidence the fact that the variation between the actual target molecule of interest and the additional molecule(s) which are also amplified may not lie at the terminal 5′ end of the variant region. That is, the variation may, in fact lie further into the variant region in the 3′ direction. If so, the primers of the present invention can, in fact be designed to provide an additional array, which is also not prohibitively or impractically large, from which a primer can be selected to provide a still further level of discrimination of the initially amplified material. Those primers, to the extent that they are utilised in the context of an additional and consecutive amplification reaction, are herein termed “leap frog primers” or “second generation primers”. Specifically, the further “leap frog” primer panel is designed such that the stem region of the leap frog primers corresponds to the stem region of the first used primer (“first generation primer”) which is adjacent to
(i) intervening bases, the number of which is equal in number to the number of universally hybridising bases which were used in the first primer and the nature of which is determined by the nature of the universally hybridising bases used in the first primer. In the situation where the intervening bases in the first primer were inosines, these intervening bases in the second primer are preferably guanines. These intervening bases are then adjacent to:
(ii) universally hybridising bases, the number of which is equal to the number of nucleotides of the recognition region of the first used primer.

Following these intervening universally hybridising bases, the primers comprise a nucleic acid recognition region which comprises the various combinations of a further two or more nucleotides, preferably 3 or 4. One of these recognition region sequences will complement the nucleotide sequence immediately 3′ to the nucleotide stretch which hybridised to the nucleic acid recognition region of the first used primer. The use of a suitably selected primer from this additional array provides an additional level of discrimination when used to probe and amplify the nucleic acid material amplified via use of the first primer. This allows one to screen for and amplify a target nucleic acid molecule utilising primers selected from two primer arrays, thereby providing a very high degree of specificity, each consisting of 256 primers in the preferred embodiment—thereby requiring a total of only 512 primers to have been generated. These arrays of primers however, are suitable for repeated use as a source of specific primers in the context of detecting any target nucleic acid molecule which falls within the class of molecules against which the arrays were generated. To the extent that the detection of a particular target sequence requires the use of a 2-step consecutive amplification process which utilises a primer directed to an initial four nucleotide stretch of the variant region followed by use of a primer directed to the next four nucleotide stretch (in the 3′ direction), one would arguably have required a primer complementary to the sequence of the first 8 nucleotide stretch of the variant region if the detection was to have been performed in a single step. To generate a pre-synthesised set of primers which hybridise at the level of 8 nucleotides and from which on appropriate primer could have been selected would have require the generation of an array comprising 4⁸ (65,536) primers—this being an entirely prohibitive number to synthesise, both in terms of practicality and cost.

It should be understood, that the use of leap frog primers is not necessarily limited to second or third round amplification processes in that they may also be utilised for initial amplification processes if their design may be suitable for a first round amplification process.

In the context of the earlier definitions provided in relation to that portion of the variant region to which the nucleic acid recognition region of the primer is directed, the above described primer set which is suitable for use subsequently to an initial amplification step is an example of a primer in which the nucleic acid recognition region is directed to a sequence of nucleotides which are not located at the terminal end of the variant region where it adjoins either the conserved region or any intervening nucleotides which may exist between the variant region and the conserved region. Rather it is directed to a sequence of nucleotides downstream of the 3′ terminal end of the variant region. In the context of these primers, which are primarily designed for use subsequently to an initial amplification step, it should be understood that the additional universally hybridising bases which are inserted into the primer for the purpose of hybridising to the nucleotides which hybridised to the nucleic acid recognition region of the first used primer fall within the definition of universally hybridising molecules “intervening said stem region and said recognition region” as hereinbefore discussed.

It should be understood that the oligonucleotide of the present invention should not be limited to the specific structure exemplified herein (being a linear, single-stranded molecule) but may extend to any suitable structural configuration which achieves the functional objectives detailed herein. For example, it may be desirable that all or part of the oligonucleotide is double stranded, comprises a looped region, such as a hairpin bend or takes the form of an open circle conformation, that is, where the nucleotide primer is substantially circular in shape but its terminal regions do not connect.

Reference to a “nucleic acid” should be understood as a reference to both deoxyribonucleic acid, ribonucleic acid, a combination of both or derivatives or analogues thereof. The nucleic acid molecules utilised in the present invention may be of any origin including naturally occurring (for example a biological sample may be utilised), recombinantly produced or synthetically produced. Preferably, said nucleic acid is deoxyribonucleic acid.

Facilitating the interaction of the nucleic acid probe with the target nucleic acid sequence may be performed by any suitable method. Those methods will be known to those skilled in the art.

In accordance with these preferred embodiments the present invention provides an array of isolated DNA primers or derivatives or analogues thereof, for use in detecting a target nucleic acid molecule which is a member of a class of nucleic acid molecules which are characterised by a specific variant region sequence, said primers comprising:
(i) a nucleic acid stem region, which stem region comprises a nucleic acid interaction site directed to a substantially conserved region of the class of which said target nucleic acid molecule is a member, or part thereof, and which substantially conserved region is located proximally to a variant region; operably linked to
(ii) a nucleic acid recognition region comprising three nucleotides
   wherein said primers comprise unique nucleic acid recognition region sequences relative to one another and wherein said primers optionally comprise at least two inosines, or analogues thereof, intervening said stem region and said recognition region.

In another preferred embodiment there is provided an array of isolated DNA primers or derivatives or analogues thereof, for use in detecting a target nucleic acid molecule which is a member of a class of nucleic acid molecules which are characterised by a specific variant region sequence, said primers comprising:
(i) a nucleic acid stem region, which stem region comprises a nucleic acid interaction site directed to a substantially conserved regions of the class of which said target nucleic acid molecule is a member, or part thereof, and which substantially conserved region is located proximally to a variant region; operably linked to
(ii) a nucleic acid recognition region comprising four nucleotides
   wherein said primers comprise unique nucleic acid recognition region sequences relative to one another and wherein said primers optionally comprise at least two inosines, or analogues thereof, intervening said stem region and said recognition region.

Reference to the nucleic acid stem region being “operably linked” to the nucleic acid recognition region should be understood as a reference to these regions being linked such that the functional objective, being hybridisation of the oligonucleotide to a target nucleic acid molecule and, optionally, amplification therefrom can be achieved. In this regard, and as detailed hereinbefore, it may be necessary that the stem region and the nucleic acid recognition region are linked via one or more universally hybridising bases. This can be necessitated by the structure of the target molecule in terms of the position of the portion of the conserved region which is the target of the stem region relative to the portion of the variant region which is the target of the recognition region, in terms of intervening nucleotides. Accordingly, where it is necessary to design an oligonucleotide of the present invention with a region of intervening universally hybridising nucleotides, it should be understood that the stem region and the recognition region are nevertheless operably linked. In terms of the means by which these regions are linked and, further, the means by which the subject oligonucleotide binds to its target molecule, these correspond to various types of interactions. In this regard, reference to “interaction” should be understood as a reference to any form of interaction such as hybridisation between complementary nucleotide base pairs or some other form of interaction such as the formation of bonds between any nucleic or non-nucleic acid portion of the primer molecule with any nucleic acid or non-nucleic acid portion of the target molecule. This type of interaction may occur via the formation of bonds such as, but not limited to, covalent bonds, hydrogen bonds, van der Wals forces or any other mechanism of interaction. Preferably, to the extent that the interaction occurs between the primer and a target molecule, said interaction is hybridisation between complementary nucleotide base pairs. All references herein to “hybridisation” between two nucleic acid molecules should be understood to encompass any form of interaction between said molecules. In order to facilitate this interaction, it is preferable that the target nucleic acid molecules are rendered partially or fully single stranded for a time and under conditions sufficient for hybridisation with a primer to occur. To the extent that the interaction occurs between the different regions of the primer molecule, these interactions will preferably occur directly between adjacent nucleotides but may also occur between any non-nucleic acid components which may form part of these regions. To the extent that the interaction does occur directly between the nucleotides, these interactions preferably take the form of covalent bonds which correspond to 3′, 5′ phosphodiester linkages.

As would be appreciated, in order to design the oligonucleotides of the present invention, it is necessary to first establish that the target nucleic acid molecule of interest is characterised by a substantially conserved region which is located proximally to a variant region and, secondly, to determine the nucleotide sequence of the conserved region to enable the design of a suitable primer array. In order to select a suitable primer for use, one does require some sequence information in relation to the variant region of the target of interest. Methods for doing so are routine and would be well known to those of skill in the art.

The development of the oligonucleotides of the present invention now provides a means of efficiently facilitating the routine screening of populations of nucleic acid molecules for the presence of a target nucleic acid molecule which is characterised by a specific variant region located proximally to a substantially conserved region. As detailed hereinbefore, this efficiency is due to the determination that target nucleic acid molecules of this type lend themselves to the generation of a relatively small number of individual primers which, in the form of an array, can achieve the hybridisation to and amplification of a target molecule of interest, within the class of molecule to which the primer was generated, exhibiting any possible variant region sequence. As detailed hereinbefore, this can be achieved through either a single step or consecutive multiple step amplification process utilising primers selected from arrays which have been designed according to the present invention. Accordingly, the present invention is particularly useful in the context of applications such as the detection, identification, quantitation and/or typing of specific genetic sequences found in biological or environmental samples such as molecular sequences of human, animal, plant, parasite, bacterial or viral origin. This includes, but is not limited to, allelic discrimination of genes, identification and/or isolation of genetic variants or mutants (for example, for the purpose of predicting patient drug responses), identification of molecules expressing specific single nucleic polymorphisms (SNPs) or for the identification and/or isolation of particular microorganism strains or the detection of mutations thereof.

An example of how such pre-synthesised primer panels could be used comes from amplification of immunoglobulin or T cell rearrangements. Rearranged immunoglobulin or T cell receptor genes are often used as molecular markers to detect low numbers of cancerous lymphocytes, eg in leukaemia, lymphoma or myeloma. Although there are some minor variations, each individual cancer in an individual patient is a clone, deriving from a single lymphocyte which has become malignant, and all cells of the tumour bear the same rearrangement. In attempting to detect low numbers of leukaemic cells, a standard approach is to use the rearranged immunoglobulin or T cell receptor gene as a molecular marker and to attempt to specifically amplify and quantify the specific rearrangement. In order to do this, it is necessary to use primers which provide a greater or lesser degree of specific amplification of the rearrangement of the malignant clone and which do not amplify other random rearrangements derived from non-malignant cells. By sequencing the particular rearrangement at diagnosis, one can select and synthesise the particular primer or primers which bind to and amplify the rearrangement from the malignant clone and which show little or no binding to other rearrangements. However this involves separate synthesis and testing for each patient, which in aggregate becomes time-consuming and expensive when many patients are being studied.

In contrast to the above situation, in which it is desired to monitor a single clone bearing a molecular marker of a sequence which has been determined and which indicates the member of the oligonucleotide panel which should be chosen and used for amplification, it is sometimes desired to study a cell population which contains many clones, each of which is defined by a different DNA sequence. If a hypervariable region is responsible for much of the DNA sequence differences, as obtains for the N regions of the rearranged immunoglobulin or T cell receptor genes, then it will be possible to define subpopulations of cells on the basis of differences in the DNA sequences at the 5′ end of the hypervariable regions. The sizes of these various subpopulations can be determined by performing multiple parallel nucleic aid amplifications with each different amplification reaction containing a different oligonucleotide from the panel. This enables an assessment of the repertoire of subpopulations within either a malignant or a non-malignant population of cells and it also enables identification of one or more subpopulations of cells which, owing to their absolute or changing size, it may be desired to follow subsequently.

These methods, which are now enabled by the development of the oligonucleotides disclosed herein, therefore have broad application including but not limited to:
(i) providing a means of monitoring the progression of a clonal population of cells in a subject. This is most likely to occur in the context of monitoring a patient in terms of the progression of a disease state or non-disease state which is characterised by the clonal expansion of a population of cells. For example, there is significant potential for the application of the method of the present invention in terms of patients suffering from malignant and non-malignant neoplasias. However, there may also be potential to apply the present invention in the context of patients suffering various forms of immunodeficiency, where one may seek to screen for the nature of specific immune cell expansion which can be mounted by that individual's immune system.
(ii) a means of diagnosing a disease condition where, for example, either the appearance or loss of a gene expressing a specific variant sequence (i.e. a mutant gene) either per se or relative to a certain threshold levels correlates to the onset of a particular condition. Such mutations may be congenital or they may have been acquired by virtue of exposure to an adverse environment factor (e.g. radiation). In addition to diagnosis, one can monitor the progress of such a disease condition.
(iii) diagnosing the presence of and/or monitoring levels of infection by a particular strain or variant of a microorganism (for example, an HIV or influenza variant), where that microorganism is characterised by a proximally located variant region/conserved region junction as hereinbefore defined. Also provided is the clinical diagnosis of a disease state or other condition which is induced by or related to the occurrence of an infection with a specific genetic form of a microorganism, such as a genetically unique bacterium, virus or parasite.
(iv) the conserved region-variant region junction sequences provide a means of marking a population of cells. For example, once these sequences have been identified, one can routinely screen populations of cells in order to identify (either qualitative and/or quantitatively) the existence of the population of cells expressing that specific marker. The method of the present invention therefore provides a relatively routine means of characterising a clonal cell population and provides for ongoing detection/monitoring applications without the need to conduct elaborate genetic analyses.

Accordingly, another aspect of the present invention is directed to a method of identifying a target nucleic acid molecule, which molecule is a member of a class of nucleic acid molecules characterised by a specific variable region sequence, in a sample said method comprising
(i) contacting said sample with an oligonucleotide as hereinbefore defined for a time and under conditions sufficient to facilitate interaction of said oligonucleotide with said target nucleic acid molecule;
(ii) amplifying said nucleic acid target; and
(iii) optionally consecutively repeating said amplification steps utilising the nucleic acid material amplified in the previous step together with a leap frog oligonucleotide; and
(iv) detecting said amplified product.

Preferably, said oligonucleotides are primers and, even more preferably, DNA primers. Yet more preferably, said nucleotide acid recognition region comprises three or four nucleotides and said universally hybridising base is inosine.

In a most preferred embodiment, said target nucleic acid molecule is a rearranged TCR or immunoglobulin variable region gene, or part thereof, and said substantially conserved portion is the 3′ end of the V gene segment.

As detailed hereinbefore reference to a “leap frog” or “second generation” oligonucleotide should be understood as a reference to the population of oligonucleotides which have been designed to provide a further level of discrimination to that afforded utilising an initial oligonucleotide. Specifically, the oligonucleotides which are initially utilised are designed, preferably, with the minimal number of nucleotides directed to the conserved region and the variant region required to either select or at least enrich for the target nucleic acid population. To the extent that the target nucleic acid population has been enriched, a further panel of oligonucleotides can be designed comprising:
(i) a stem region;
(ii) a region of specific bases which hybridises to the bases in the amplified target corresponding to the universally hybridising bases of the first primer;
(iii) universally hybridising bases which correspond to the bases of recognition sequence of the primer and
(iv) a recognition sequence.

The “leap frog” oligonucleotide is then designed with a recognition region directed to the nucleotide sequence 3′ to the sequence recognised by the recognition region of the first oligonucleotide.

Methods for achieving primer directed amplification are well known to those of skill in the art. In a preferred method, said amplification is polymerase chain reaction, NASBA or strand displacement amplification.

Reference to a “sample” should be understood as a reference to either a biological or a non-biological sample. Examples of non-biological samples includes, for example, the nucleic acid products of synthetically produced nucleic acid populations. Reference to a “biological sample” should be understood as a reference to any sample of biological material derived from an animal, plant or microorganism (including cultures of microorganism) such as, but not limited to, cellular material, blood, mucus, faeces, urine, tissue biopsy specimens, fluid which has been introduced into the body of an animal and subsequently removed (such as, for example, the saline solution extracted from the lung following lung lavage or the solution retrieved from an enema wash), plant material or plant propagation material such as seeds or flowers or a microorganism colony. The biological sample which is tested according to the method of the present invention may be tested directly or may require some form of treatment prior to testing. For example, a biopsy sample may require homogenisation prior to testing. For example, a biopsy sample may require homogenisation prior to testing or it may require sectioning for in situ testing. Further, to the extent that the biological sample is not in liquid form, (if such form is required for testing) it may require the addition of a reagent, such as a buffer, to mobilise the sample.

To the extent that the target molecule is present in a biological sample, the biological sample may be directly tested or else all or some of the nucleic acid material present in the biological sample may be isolated prior to testing. It is within the scope of the present invention for the target nucleic acid molecule to be pre-treated prior to testing, for example, inactivation of live virus or being run on a gel. It should also be understood that the biological sample may be freshly harvested or it may have been stored (for example by freezing) prior to testing or otherwise treated prior to testing (such as by undergoing culturing).

Reference to “contacting” the sample with the primer should be understood as a reference to facilitating the mixing of the primer with the sample such that interaction (for example, hybridisation) can occur. Means of achieving this objective would be well known to those of skill in the art.

The choice of what type of sample is most suitable for testing in accordance with the method disclosed herein will be dependent on the nature of the situation, such as the nature of the condition being monitored. For example, in a preferred embodiment a neoplastic condition is the subject of analysis. If the neoplastic condition is a lymphoid leukaemia, a blood sample, lymph fluid sample or bone marrow aspirate would likely provide a suitable testing sample. Where the neoplastic condition is a lymphoma, a lymph node biopsy or a blood or marrow sample would likely provide a suitable source of tissue for testing. Consideration would also be required as to whether one is monitoring the original source of the neoplastic cells or whether the presence of metastases or other forms of spreading of the neoplasia from the point of origin is to be monitored. In this regard, it may be desirable to harvest and test a number of different samples from any one mammal. Choosing an appropriate sample for any given detection scenario would fall within the skills of the person of ordinary skill in the art.

The term “mammal” to the extent that it is used herein includes humans, primates, livestock animals (e.g. horses, cattle, sheep, pigs, donkeys), laboratory test animals (e.g. mice, rats, rabbits, guinea pigs), companion animals (e.g. dogs, cats) and captive wild animals (e.g. kangaroos, deer, foxes). Preferably, the mammal is a human or a laboratory test animal. Even more preferably the mammal is a human.

The method of this aspect of the present invention provides a means for both detecting the presence of a target nucleic acid molecule of interest and, optionally, quantifying and/or isolating that target. Accordingly, one is provided with means of either enriching or purifying a target nucleic acid population of interest for any purpose, such as further analysis of the target.

It should be understood that the execution of the method of the present invention is not intended to be limited to the specific means detailed herein since the design and application of such means would be well within the skill of the person of skill in the art.

Another aspect of the present invention provides a method of detecting and/or monitoring a clonal population of cells in a mammal, which clonal cells are characterised by a target nucleic acid molecule which is a member of a class of nucleic acid molecules characterised by a specific variant region sequence, said method comprising:
(i) contacting the nucleic acid material of a biological sample derived from a mammal with an oligonucleotide as hereinbefore defined for a time and under conditions sufficient to facilitate interaction of said oligonucleotide with said target nucleic acid molecule;
(ii) amplifying said nucleic acid target;
(iii) optionally consecutively repeating said amplification steps utilising the nucleic acid material amplified in the preceding step together with a leap frog oligonucleotide, and
(iv) detecting said amplified product.

Preferably, said oligonucleotides are primers and, even more preferably, DNA primers. Yet more preferably, said nucleotide acid recognition region comprises three or four nucleotides and said universally hybridising base is inosine.

In a most preferred embodiment, said target nucleic acid molecule is a rearranged TCR or immunoglobulin variable region gene, or part thereof, and said substantially conserved portion is the 3′ end of the V gene segment.

Reference to “cells” should be understood as a reference to all forms of cells from any species and to mutants or variants thereof. Without limiting the present invention to any one theory or mode of action, a cell may constitute an organism (in the case of unicellular organisms) or it may be a subunit of a multicellular organism in which individual cells may be more or less specialised (differentiated) for particular functions. All living organisms are composed of one or more cells. The subject cell may form part of the biological sample which is the subject of testing in a syngeneic, allogeneic or xenogeneic context. A syngeneic process means that the clonal cell population and the biological sample within which that clonal population exists share the same MHC genotype. This will most likely be the case where one is screening for the existence of a neoplasia in an individual, for example. An “allogeneic” process is where the subject clonal population in fact expresses a different MHC to that of the individual from which the biological sample is harvested. This may occur, for example, where one is screening for the proliferation of a transplanted donor cell population (such as an immunocompetent bone marrow transplant) in the context of a condition such as graft versus host disease. A “xenogeneic” process is where the subject clonal cells are of an entirely different species to that of the subject from which the biological sample is derived. This may occur, for example, where a potentially neoplastic donor population is derived from xenogeneic transplant.

“Variants” of the subject cells include, but are not limited to, cells exhibiting some but not all of the morphological or phenotypic features or functional activities of the cell of which it is a variant. “Mutants” includes, but is not limited to, cells which have been naturally or non-naturally modified such as cells which are genetically modified.

By “clonal” is meant that the subject population of cells has derived from a common cellular origin. For example, a population of neoplastic cells is derived from a single cell which has undergone transformation at a particular stage of differentiation. In this regard, a neoplastic cell which undergoes further nuclear rearrangement or mutation to produce a genetically distinct population of neoplastic cells is also a “clonal” population of cells, albeit a distinct clonal population of cells. In another example, a T or B lymphocyte which expands in response to an acute or chronic infection or immune stimulation is also a “clonal” population of cells within the definition provided herewith. In yet another example, the clonal population of cells is a clonal microorganism population, such as a drug resistant clone which has arisen within a larger microorganismal population. Preferably, the subject clonal population of cells is a neoplastic population of cells or a clonal immune cell population.

Preferably said clonal cells are a population of clonal lymphoid cells.

It should be understood that reference to “lymphoid cell” is a reference to any cell which has rearranged at least one germ line set of immunoglobulin or TCR variable region gene segments. The immunoglobulin variable region encoding genomic DNA which may be rearranged includes the variable regions associated with the heavy chain or the K or X light chain while the TCR chain variable region encoding genomic DNA which may be rearranged include the α, β, γ and δ chains. In this regard, a cell should be understood to fall within the scope of the “lymphoid cell” definition provided the cell has rearranged the variable region encoding DNA of at least one immunoglobulin or TCR gene segment region. It is not necessary that the cell is also transcribing and translating the rearranged DNA. In this regard, “lymphoid cell” includes within its scope, but is in no way limited to, immature T and B cells which have rearranged the TCR or immunoglobulin variable region gene segments but which are not yet expressing the rearranged chain (such as TCR⁻ thymocytes) or which have not yet rearranged both chains of their TCR or immunoglobulin variable region gene segments. This definition further extends to lymphoid-like cells which have undergone at least some TCR or immunoglobulin variable region rearrangement but which cell may not otherwise exhibit all the phenotypic or functional characteristics traditionally associated with a mature T cell or B cell. Accordingly, the method of the present invention can be used to monitor neoplasias of cells including, but not limited to, lymphoid cells at any differentiative stage of development, activated lymphoid cells or non-lymphoid/lymphoid-like cells provided that rearrangement of at least part of one variable region gene region has occurred. It can also be used to monitor the clonal expansion which occurs in response to a specific antigen.

It should also be understood that although it is preferable that the rearrangement of at least one variable region gene region has been completed, the method of the present invention is nevertheless applicable to monitoring neoplastic cells which exhibit only partial rearrangement. For example, a B cell which has only undergone the DJ recombination event is a cell which has undergone only partial rearrangement. Complete rearrangement will not be achieved until the DJ recombination segment has further recombined with a V segment. The method of the present invention can therefore be designed to detect the partial or complete variable region rearrangement of one TCR or immunoglobulin chain utilising a reference molecule complementary to this marker sequence or, for example, if greater specificity is required and the neoplastic cell has rearranged the variable region of both TCR or immunoglobulin chains, primer molecules directed to both forms of rearrangement can be utilised.

Reference to a “neoplastic cell” should be understood as a reference to a cell exhibiting abnormal “growth”. The term “growth” should be understood in its broadest sense and includes reference to proliferation. In this regard, an example of abnormal cell growth is the uncontrolled proliferation of a cell. The uncontrolled proliferation of a lymphoid cell may lead to a population of cells which take the form of either a solid tumour or a single cell suspension (such as is observed, for example, in the blood of a leukemic patient). A neoplastic cell may be a benign cell or a malignant cell. In a preferred embodiment, the neoplastic cell is a malignant cell. In this regard, reference to a “neoplastic condition” is a reference to the existence of neoplastic cells in the subject mammal. Although “neoplastic lymphoid condition” includes reference to disease conditions which are characterised by reference to the presence of abnormally high numbers of neoplastic cells such as occurs in leukemias, lymphomas and myelomas, this phrase should also be understood to include reference to the circumstance where the number of neoplastic cells found in a mammal falls below the threshold which is usually regarded as demarcating the shift of a mammal from an evident disease state to a remission state or vice versa (the cell number which is present during remission is often referred to as the “minimal residual disease”). Still further, even where the number of neoplastic cells present in a mammal falls below the threshold detectable by the screening methods utilised prior to the advent of the present invention, the mammal is nevertheless regarded as exhibiting a “neoplastic condition”.

As detailed hereinbefore, the development of the primers of the present invention have now facilitated the development of diagnostic and monitoring applications which are based on achieving high use specific nucleic acid discrimination utilising one or more arrays of relatively modest numbers of presynthesised oligonucleotide primers. This has widespread application in, inter alia, disease monitoring, diagnosis and prognosis, genetic profiling and the detection of specific microorganisms infections.

Accordingly, still another aspect of the present invention is directed to a method for diagnosis of the onset of or a predisposition to the onset of a disease condition or for monitoring or prognosing the progression of a disease condition in a mammal, which condition is characterised by the presence or change in the level of a target nucleic acid molecule, or clonal cell population characterised by a target nucleic acid molecule, which molecule is a member of a class nucleic acid molecule characterised by a specific variant region sequence, said method comprising:
(i) contacting a sample derived from said mammal with an oligonucleotide as hereinbefore defined, for a time and under conditions sufficient to facilitate interaction of said oligonucleotide with said target nucleic acid molecule;
(ii) amplifying said nucleic acid target;
(iii) optionally consecutively repeating said amplification steps utilising the nucleic acid material amplified in the preceding step together with a leap frog oligonucleotide; and
(iv) detecting said amplified product.

Disease conditions suitable for analysis in this regard are any lymphoid malignancies such as acute lymphoblastic leukaemia, chronic lymphocytic leukaemia, non-Hodgkin's lymphoma and myeloma. Monitoring of minimal residual disease is of importance in all of these conditions. Other situations in which this method is applicable include monitoring of bacterial or viral infections, particularly those in which there is a great deal of genetic variation. Human or animal retroviral infections such as HIV are just one example.

With respect to this aspect of the present invention, reference to “monitoring” should be understood as a reference to testing the subject for the presence or level of the subject clonal population of cells after initial diagnosis of the existence of said population. “Monitoring” includes reference to conducting both isolated one off tests or a series of tests over a period of days, weeks, months or years. The tests may be conducted for any number of reasons including, but not limited to, predicting the likelihood that a mammal which is in remission will relapse, monitoring the effectiveness of a treatment protocol, checking the status of a patient who is in remission, monitoring the progress of a condition prior to or subsequently to the application of a treatment regime, in order to assist in reaching a decision with respect to suitable treatment or in order to test new forms of treatment. The method of the present invention is therefore useful as both a clinical tool and a research tool.

Preferably, said condition is a neoplasia and even more preferably a lymphoid neoplasia.

Yet another aspect of the present invention is directed to a kit for facilitating the identification of a target nucleic acid molecule, said kit comprising compartments adapted to contain any one or more of the oligonucleotide primers as hereinbefore defined, reagents useful for facilitating interaction of said primer with the target nucleic acid molecule and reagents useful for enabling said interaction to result in amplification of said nucleic acid target. Further compartments may also be included, for example, to receive biological or non-biological samples.

Further features of the present invention are more fully described in the following non-limiting examples.

### EXAMPLE 1

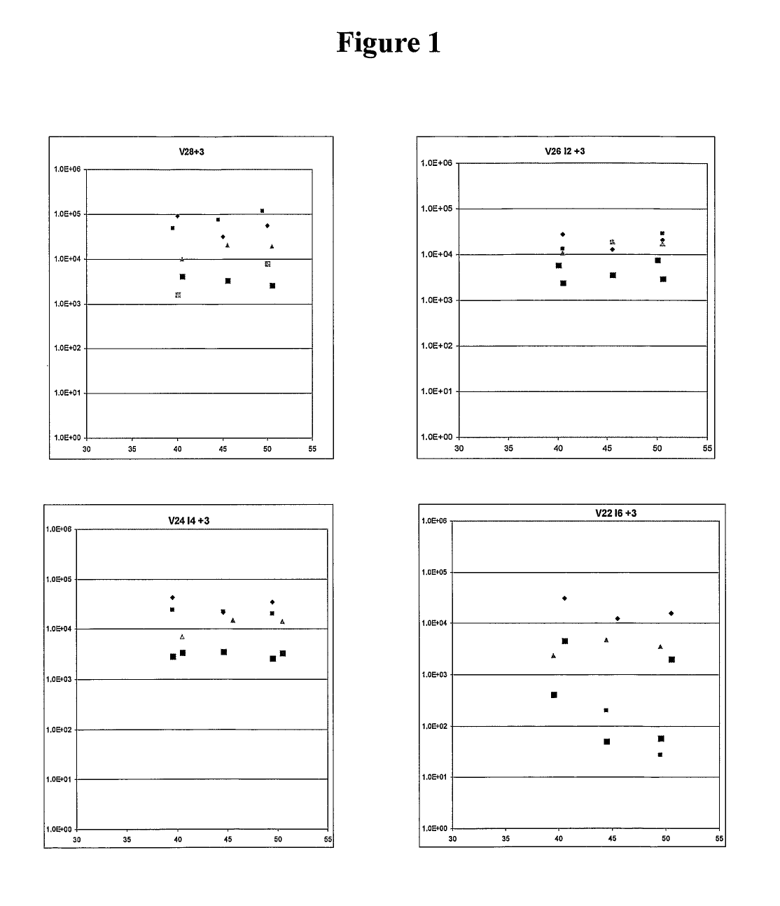

Study of DNA samples from 5 patients with ALL. The effect of inosine number was studied by using primers 31 bases in length and, proceeding from the 3′ to 5′ end, having: 3 bases of perfect match (to the first 3 variable bases of the N region); 0,2,4 or 6 inosines and; 28, 26, 24 or 22 bases of perfect match. The effect of annealing temperature was also studied. The end-point was the amplification achieved by 20 cycles of PCR. Amplification fell below 10⁴ when primers containing 6 inosines were used. In this experiment, temperature had no effect but an effect was seen in some other experiments. The final conditions when using primers directed at 3 variable bases were: primers containing 4 inosines at an annealing temperature of 43 Celsius. Another experiment showed that a primer directed towards 4 variable bases was tolerant to 6 or 8 inosines at 43 Celsius (FIG. 1).

### EXAMPLE 2

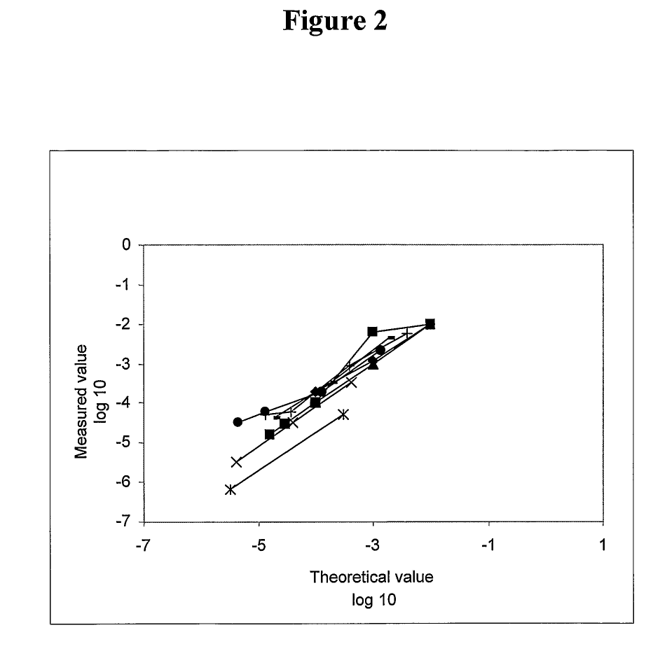

Results of detection of low numbers of leukemic cells in 7 experiments in which the leukemic cells were mixed in various proportions with normal peripheral blood cell. Specific amplification of the leukemic cells was achieved by 3 sequential PCRs, the second of which involved a primer containing 4 inosines followed by 3 bases at the 3′ end which matched the first 3 bases of the N region. There is excellent correlation between the observed results and the theoretical results as expected from the mixtures that were made (FIG. 2).

### EXAMPLE 3

| | | ino- | final | | | |
|---|---|---|---|---|---|---|
| Patient/Expt | | sines | 3′ bases | Site | Result 1 | Result 2 |
| 393/03 | | | | | | |
| | 394 | 4 | TTG | A1 | <1.2 × 10 −6 | 3.7 × 10 −5 |
| | 395 | | | A2 | <1.2 × 10 −6 | 4.6 × 10 −5 |
| | 396 | | | A3 | 3.71 × 10 −6 | <2.2 × 10 −6 |
| | 397 | | | B1 | 9.7 × 10 −5 | 1.8 × 10 −5 |
| | 398 | | | B2 | <1.2 × 10 −6 | 5.04 × 10 −5 |
| 399/03 | | 4 | CGG | | | |
| | 400 | | | A1 | 8.1 × 10 −4 | 3.3 × 10 −4 |
| | 401 | | | A2 | 2.43 × 10 −4 | 1.22 × 10 −4 |
| | 402 | | | A3 | 4.47 × 10 −4 | 3.89 × 10 −4 |
| | 403 | | | B1 | 2.53 × 10 −4 | 1.46 × 10 −4 |
| | 404 | | | B2 | 3.4 × 10 −4 | 3.3 × 10 −4 |
| 405/03 | | 4 | TTT | | | |
| | 406 | | | A1 | 1.3 × 10 −6 | 1.84 × 10 −5 |
| | 407 | | | A2 | 9.6 × 10 −6 | <2.2 × 10 −7 |
| | 408 | | | A3 | 6.52 × 10 −6 | <3.3 × 10 −7 |
| | 409 | | | B1 | 3.1 × 10 −5 | 2.9 × 10 −5 |
| | 410 | | | B2 | 1.2 × 10 −5 | 7.8 × 10 −6 |
| 411/03 | | 4 | CGT | | | |
| | 412 | | | A1 | 3.22 × 10 −7 | 3.07 × 10 −7 |
| | 413 | | | A2 | <4 × 10 −7 | <4 × 10 −7 |
| | 414 | | | A3 | <4 × 10 −7 | <4 × 10 −7 |
| | 415 | | | B1 | <4 × 10 −7 | <4 × 10 −7 |
| | 416 | | | B2 | <4 × 10 −7 | <4 × 10 −7 |
| 417/03 | | 4 | TCAA | | | |
| | 418 | | | A1 | 1.02 × 10 −2 | 4.1 × 10 −3 |
| | 419 | | | A2 | 1.05 × 10 −2 | 4.46 × 10 −3 |
| | 420 | | | A3 | 3.51 × 10 −2 | 1.3 × 10 −2 |
| | 421 | | | B1 | 1.41 × 10 −2 | 6.3 × 10 −2 |
| | 422 | | | B2 | 1.04 × 10 −2 | 1.20 × 10 −2 |
| 102/04 | | 6 | TTAA | | | |
| | 90 | | | A1 | <2.14 × 10 −6 | <3.02 × 10 −7 |
| | 91 | | | A2 | 1.76 × 10 −5 | <2.19 × 10 −7 |
| | 92 | | | A3 | <2.4 × 10 −6 | <2.84 × 10 −7 |
| | 93 | | | A4 | <2.5 × 10 −6 | <2.2 × 10 −7 |
| | 94 | | | A5 | <1.5 × 10 −6 | <2.2 × 10 −7 |
| 228/04 | | 6 | CCGA | | | |
| | 299 | | | A1 | 3.9 × 10 −5 | 1.56E−07 |
| | 230 | | | A2 | 1.8 × 10 −5 | 1.66E−06 |
| | 231 | | | A3 | 5.5 × 10 −5 | 1.85E−06 |
| | 232 | | | B1 | 5.28 × 10 −5 | 5.72E−06 |
| | 233 | | | B2 | 1.39 × 10 −5 | 9.98E−06 |
| 179/04 | | 6 | TGGA | | | |
| | 180 | | | A1 | 2.10E−04 | |
| | 181 | | | A2 | 1.85E−04 | |
| | 182 | | | A3 | 8.49E−05 | |
| | 183 | | | B1 | 1.99E−04 | 6.81E−05 |
| | 184 | | | B2 | 8.61E−05 | 5.40E−06 |
| 222/04 | | 6 | AAAA | | | |
| | 223 | | | A1 | 3.91E−05 | 2.49E−05 |
| | 224 | | | A2 | 1.73E−05 | 2.26E−05 |
| | 225 | | | A3 | 3.24E−05 | 1.57E−05 |
| | 226 | | | B1 | 7.49E−06 | 4.86E−06 |
| | 227 | | | B2 | 2.58E−05 | 3.94E−05 |
| 216/04 | | 6 | GATA | | | |
| | 217 | | | A1 | 7.76E−04 | |
| | 218 | | | A2 | 6.24E−04 | |
| | 219 | | | A3 | 2.75E−04 | |
| | 220 | | | B1 | 2.05E−04 | |
| | 221 | | | B2 | 4.70E−04 | |

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations of any two or more of said steps or features.

1. An array of isolated nucleic acid molecules or derivatives or analogues thereof, for use in detecting a target nucleic acid molecule which is a member of a class of nucleic acid molecules and which is characterised by a specific variant region, said nucleic acid molecules comprising:
(i) a nucleic acid stem region, which stem region comprises a nucleic acid interaction site directed to a substantially conserved region of the class of which said target nucleic acid molecule is a member, or part thereof and which substantially conserved region is located proximally to a variant region; operably linked to
(ii) a nucleic acid recognition region comprising at least two nucleotides wherein said nucleic acid molecules comprise unique nucleic acid recognition region sequences relative to one another and wherein said nucleic acid molecules optionally comprise one or more universally hybridising bases, or analogues thereof, intervening said stem region and said recognition region.

2. An isolated nucleic acid molecule or derivative or analogue thereof, for use in detecting a target nucleic acid molecule which is a member of a class of nucleic acid molecules and which is characterised by a specific variant region, said nucleic acid molecule comprising:
(i) a nucleic acid stem region, which stem region comprises a nucleic acid interaction site directed to a substantially conserved region of the class of which said target nucleic acid molecule is a member, or part thereof and which substantially conserved region is located proximally to a variant region; operably linked to
(ii) a nucleic acid recognition region comprising at least two nucleotides
wherein said nucleic acid molecule optionally comprises one or more universally hybridising bases, or analogues thereof, intervening said stem region and said recognition region.

3. The array according to claim 1 or molecule according to claim 2 wherein said recognition region is operably linked to the 3′ end of the nucleic acid stem region of said isolated nucleic acid molecule.

4. The array according to claim 1 or 3 or molecule according to claim 2 or 3 wherein said class of nucleic acid molecules is the rearranged genomic immunoglobulin genes.

5. The array or molecule according to claim 4 wherein said rearranged genomic immunoglobulin gene is the heavy chain gene.

6. The array or molecule according to claim 4 wherein said rearranged genomic immunoglobulin gene is the light chain gene.

7. The array according to claim 1 or 3 or molecule according to claim 2 or 3 wherein said class of nucleic acid molecules is the rearranged genomic T cell receptor genes.

8. The array or molecule according to claim 7 wherein said rearranged genomic T cell receptor gene is the α chain gene.

9. The array or molecule according to claim 7 wherein said rearranged genomic T cell receptor gene is the β chain gene.

10. The array or molecule according to claim 7 wherein said rearranged genomic T cell receptor gene is the y chain gene.

11. The array or molecule according to claim 7 wherein said rearranged genomic T cell receptor gene is the 8 chain gene.

12. The array or molecule according to any one of claims 4 to 11 wherein said nucleic acid interaction site is directed to a substantially conserved portion of the 5′ end of the antisense strand of the V gene segment.

13. The array or molecule according to claim 12 wherein said conserved portion of the V gene segment is a conserved portion of the F_{R3I} or F_{R3II} segment.

14. The array or molecule according to any one of claims 4, 5, 7, 9, 10 or 11 wherein said nucleic acid interaction site is directed to a substantially conserved portion of the 5′ end of the antisense strand of the D gene segment.

15. The array or molecule according to any one of claims 4 to 11 wherein said nucleic acid interaction site is directed to a substantially conserved portion of the 5′ end of the sense strand of the J gene segment.

16. The array or molecule according to any one of claims 4, 5, 7, 9, 10 or 11 wherein said nucleic acid interaction site is directed to a substantially conserved portion of the 5′ end of the sense strand of the D gene segment.

17. The array or molecule according to any one of claims 1 to 16 wherein said stem region is from 5 to 50 nucleotides in length.

18. The array or molecule according to claim 17 wherein said stem region is from 10 to 40 nucleotides in length.

19. The array or molecule according to claim 18 wherein said stem region is from 15 to 35 nucleotides in length.

20. The array or molecule according to claim 19 wherein said stem region is from 20 to 35 nucleotides in length.

21. The array or molecule according to claim 20 wherein said stem region is 20, 21, 22, 23, 24, 25, 26, 27, 28, 29, 30, 31, 32, 33, 34 or 35 nucleotides in length.

22. The array or molecule according to any one of claims 17 to 21 wherein said nucleic acid recognition region is 2, 3, 4, 5 or 6 nucleotides in length.

23. The array or molecule according to claim 22 wherein said nucleic acid recognition region is 3 nucleotides in length.

24. The array or molecule according to claim 22 wherein said nucleic acid recognition region is 4 nucleotides in length.

25. The array or molecule according to any one of claims 1 to 24 wherein said isolated nucleic acid molecule is an isolated oligonucleotide.

26. The array or molecule according to claim 25 wherein said oligonucleotide is an oligonucleotide primer.

27. The array or molecule according to claim 26 wherein said oligonucleotide primer is DNA.

28. The array or molecule according to claim 27 wherein said universally hybridising base is inosine.

29. The array or molecule according to claim 28 wherein said isolated nucleic acid molecule comprises at least two inosines.

30. A second generation isolated nucleic acid molecule or derivative or analogue thereof, for use in detecting a target nucleic acid molecule which is a member of a class of nucleic acid molecules and which is characterised by a specific variant region, said nucleic acid molecule comprising:
(i) a stem region which corresponds to the stem region of the first generation nucleic acid molecule of any one of claims 1 to 28; operably linked to
(ii) an intervening base region, which intervening base region is directed to the universally hybridising bases of the first generation nucleic acid molecule and which intervening base region is operably linked to
(iii) a region of universally hybridising bases directed to the nucleic acid recognition region of said first generation nucleic acid molecule and which region of universally hybridising bases is operably linked to
(iv) a nucleic acid recognition region comprising at least two nucleotides directed to the nucleotide sequence 3′ to the sequence to which the recognition region of the first generation nucleic acid molecule is directed.

31. An array of second generation nucleic acid molecules according to claim 30 wherein said nucleic acid molecules comprise unique nucleic acid recognition sequences relative to one another.

32. The array according to any one of claims 1, 3 to 29 or 31 wherein said array comprises two or more nucleic acid molecules comprising unique nucleic acid recognition sequences relative to one another.

33. A method for identifying a target nucleic acid molecule in a sample, which molecule is a member of a class of nucleic acid molecules characterised by a specific variant region sequence, said method comprising (i) contacting said sample with a nucleic acid molecule according to any one of claims 1 to 29 for a time and under conditions sufficient to facilitate interaction of said nucleic acid molecule with said target nucleic acid molecule;
(ii) amplifying said nucleic acid target; and
(iii) optionally consecutively repeating said amplification steps utilising the nucleic acid material amplified in the preceding step together with a second generation nucleic acid molecule according to claim 30 to 32; and
(iv) detecting said amplified product.

34. A method for detecting and/or monitoring a clonal population of cells in a mammal, which clonal cells are characterised by a target nucleic acid molecule which is a member of a class of nucleic acid molecules characterised by a specific variant region sequence, said method comprising:
(i) contacting the nucleic acid material of a biological sample derived from a mammal with a nucleic acid molecule according to any one of claims 1 to 29 for a time and under conditions sufficient to facilitate interaction of said nucleic acid molecule with said target nucleic acid molecule;
(ii) amplifying said nucleic acid target;
(iii) optionally consecutively repeating said amplification steps utilising the nucleic acid material amplified in the preceding step together with a second generation nucleic acid molecule according to claim 30 to 32; and
(iv) detecting said amplified product.

35. The method according to claim 34 wherein said clonal population of cells is a neoplastic population of cells.

36. The method according to claim 35 wherein said neoplastic population of cells is a population of neoplastic lymphoid cells.

37. A method for the diagnosis of the onset of or a predisposition to the onset of a disease condition or for monitoring or prognosing the progression of a disease condition in a mammal, which condition is characterised by the presence or change in the level of a target nucleic acid molecule, or clonal cell population characterised by a target nucleic acid molecule, which molecule is a member of a class nucleic acid molecules characterised by a specific variant region sequence, said method comprising:
(i) contacting a sample derived from said mammal with a nucleic acid molecule according to any one of claims 1 to 29, for a time and under conditions sufficient to facilitate interaction of said nucleic acid molecule with said target nucleic acid molecule;
(ii) amplifying said nucleic acid target;
(iii) optionally consecutively repeating said amplification steps utilising the nucleic acid material amplified in the preceding step together with a second generation nucleic acid molecule according to claim 30 to 32; and
(iv) detecting said amplified product.

38. The method according to claim 37 wherein said disease condition is a neoplastic condition and said clonal population of cells is a neoplastic population of cells.

39. The method according to claim 38 wherein said neoplastic population of cells is a population of lymphoid cells.

40. The method according to claim 37 wherein said condition is a microorganism infection and said microorganism is a particular species or variant.

41. The method according to any one of claims 33 to 40 wherein said nucleic acid molecule and second generation nucleic acid molecule are oligonucleotides.

42. The method according to claim 41 wherein said oligonucleotide is a primer.

43. The method according to claim 42 wherein said primer is a DNA primer.

44. The method according to claim 43 wherein the nucleic acid recognition region of said DNA primer comprises 3 nucleotides.

45. The method according to claim 43 wherein the nucleic acid recognition region of said DNA primer comprises 4 nucleotides.

46. The method according to any one of claims 33 to 39 or 41 to 45 wherein said target nucleic acid molecule is a rearranged genomic T cell receptor gene.

47. The method according to any one of claims 33 to 39 or 41 to 45 wherein said target nucleic acid molecule is a rearranged genomic immunoglobulin receptor gene.

48. The method according to claim 46 or 47 wherein the substantially conserved portion is the 3′ end of the V gene segment.

49. A kit for facilitating the identification of a target nucleic acid molecule, said kit comprising compartments adapted to contain any one or more of the nucleic acid molecules according to claims 1 to 32, reagents useful for facilitating interaction of said nucleic acid molecule with the target nucleic acid molecule and reagents useful for enabling said interaction to result in amplification of said nucleic acid target.

50. The kit according to claim 49 when used in the method of any one of claims 33 to 48.
